(19) United States
(12) Patent Application Publication
(10) Pub. No.: US 2007/0053846 A1
(43) Pub. Date: Mar. 8, 2007

(54) Dry coating and downstream processing of cohesive powders

(76) Inventors: Rajesh N. Dave, Short Hills, NJ (US); Robert Pfeffer, Scottsdale, AZ (US); Jun Yang, Kearny, NJ (US); Yuhua Chen, Newark, NJ (US)

Correspondence Address: McCARTER & ENGLISH, LLP;Attn: Basam E. Nabulsi, Financial Centre, Suite 304A, 695 East Main Street, Stamford, CT, 06901-2138, US

(21) Appl. No.: 11/512,453

(22) Filed: Aug. 30, 2006

Related U.S. Application Data
(60) Provisional application No. 60/712,910, filed on Aug. 31, 2005.

(51) Int. Cl. A61K 9/28; A61K 9/14

(52) U.S. Cl. 424/46; 427/2.14

(57) ABSTRACT

The present disclosure is directed to systems and methods for dry particle coating of cohesive powders, and to the dry coated particles/powders produced thereby. The present disclosure is further directed to systems and methods for dry coating of cohesive particles, particularly nanosized particles, to provide enhanced flowability and other advantageous physical and/or functional properties. The disclosed systems and methods offer downstream processing advantages, e.g., for purposes of subsequent fluidization, coating, granulation and/or other particle processing operations, and have applicability in wide ranging industries, including specifically paint-related applications, pharmaceutical applications, food-related applications, cosmetic applications, defense-related applications, electronics-related applications, toner and ink-related applications, and the like.

### CROSS-REFERENCE TO RELATED APPLICATION

The present application claims the benefit of a co-pending provisional patent application entitled “Dry Particle Coating of Cohesive Powders and Associated Methods,” which was filed on Aug. 31, 2006, and assigned Ser. No. 60/712,910. The entire contents of the foregoing provisional patent application are incorporated herein by reference.

1. Technical Field

### BACKGROUND

The present disclosure is directed to systems and methods for dry coating and downstream processing of cohesive powders, and to the dry coated particles/powders and downstream powders/products produced thereby. The present disclosure is further directed to systems and methods for dry coating of cohesive particles, particularly with nanosized particles, to provide enhanced flowability and other advantageous physical and/or functional properties. The disclosed systems and methods offer downstream processing advantages, e.g., for purposes of subsequent fluidization, coating, granulation, mixing, and/or other particle processing operations, and have applicability in wide ranging industries, including specifically paint-related applications, pharmaceutical applications, food-related applications, cosmetic applications, defense-related applications, electronics-related applications, toner and ink-related applications, and the like.

2. Background Art

Handling and processing of fine particles in the dry state, e.g., smaller than ˜30 μm, is a challenging industrial issue. Generally, such powders exhibit poor flowability properties due to the cohesive forces that arise between the particles, e.g., based on Van der Waals attractive forces. Vibration and sound waves are two processing techniques that are routinely utilized in an effort to overcome such cohesive forces and improve flowability. The addition of one or more flow agents to a powder system has also been employed in an effort to reduce such cohesive forces. For example, a small amount of fumed silica “guest” particles mixed or blended together with the cohesive “host” particles can improve flowability. However, most flow agents (e.g., fumed silica) consist of very fine particles that have a strong tendency to form agglomerates. Thus, de-agglomeration and dispersion of the flow agent is important for achieving desired coating effects (rather than simple mixing or blending) and achieving advantageous flowability properties.

Researchers from the New Jersey Institute of Technology have investigated dry coating techniques. For example, dry particle coating concepts and techniques are described by Pfeffer et al. in an article entitled “Synthesis of engineered particulates with tailored properties using dry particle coating,” Powder Technology 117 (2001), pgs. 40-67. As described in the foregoing article, dry particle coating may be used to create new-generation materials by combining different powders having different physical and chemical properties to form composites. The new-generation materials described by Pfeffer et al. exhibit unique functionalities and/or improved characteristics relative to known materials. In particular, Pfeffer et al. describe techniques for mechanically coating materials of relatively large size (1-200 μm) with fine submicron particles in the absence of a liquid (e.g., a solvent, binder or water). The contents of the foregoing article are incorporated herein by reference.

Previous development efforts have also focused on “dry blending” techniques and processes to enhance the flowability of cohesive particles. Thus, for example, U.S. Pat. No. 6,833,185 to Zhu describes dry blending of fluidization additives with cohesive powders. The fluidization additives are characterized by a smaller size and lesser mean “apparent” particle density relative to the cohesive fine powders to which they are added. Of note, the “dry blending” step according to the Zhu patent merely blends the fluidization additives with the underlying cohesive powders and does not affect a “coating” of the additives onto (or with respect to) the underlying cohesive powders, as would be the case in a “dry coating” step.

Additional background teachings of note include:
An article entitled “Coat of Many Properties,” Process Engineering, October 2003, Vol. 84, No. 10, pg. 23, which discusses dry coating to form an ordered mixture (termed “mechanofusion”). Mechanofusion combines high shear and compression to create an ordered mixture through dry coating to improve flowability.
U.S. Pat. No. 5,955,530 to Inoue, which discloses a dry blending technique to form a powder coating composition of thermosetting resin compositions having particular particle size properties/characteristics.
U.S. Pat. No. 5,336,309 to Noguchi, which discloses a high speed stirring technique (in the absence of a liquid) to form a flaky pigment having a mean particle size from 5-60 μm.
U.S. Pat. No. 5,470,893 to Sinclair-Day, which discloses a process for preparation of a powder coating composition that includes a primary film-forming component. The process involves comminuting the components and mixing and agglomerating them to produce composite particles such that the composition may be fluidized in air.

Although prior art efforts have attempted to address flowability and related issues associated with cohesive powder systems, a need remains for enhanced processing techniques and methodologies that facilitate handling/processing of cohesive powder systems in the dry state. For example, a need remains for processing techniques and methodologies that are reliable and that generate homogeneous powder systems. Moreover, a need remains for enhanced processing techniques and methodologies that permit reduced “additive” levels while achieving advantageous fluidization benefits. These and other needs and benefits are achieved through the systems and methods disclosed herein.

### SUMMARY OF DISCLOSURE

According to the present disclosure, advantageous systems and methods for reducing cohesive forces associated with cohesive powders are provided. The disclosed systems and methods include a dry coating step, whereby fine, nanosized particles are dry coated onto cohesive powders. The fine, nanosized particles are generally uniformly dispersed over the surface of the cohesive powders, and generally define a very sparse layer thereon. Typically, the fine, nanosized particles are coated onto the cohesive powders at levels of about 0.04 to about 2 wt. %. Despite the relatively limited amount of fine, nanosized particles introduced to the cohesive powder system, the cohesive forces associated with the cohesive powder are generally reduced by at least an order of magnitude or better.

The disclosed systems and methods achieve an advantageous reduction in cohesive forces for cohesive powders. The cohesive powders are generally characterized, in whole or in part, by particles falling within Geldart Group “C”. However, through the dry coating technique of the present disclosure, such particles are advantageously converted to Geldart Group “A” and/or Geldart Group “B” particles, thereby facilitating subsequent fluidization or other downstream processing thereof. Of particular note, by reducing the cohesive forces of cohesive powders as described herein, the disclosed systems and methods facilitate the processing of smaller particles than otherwise possible in fluidized beds. Thus, fluidized powder systems of the present disclosure may be subjected to various treatment regimens within the fluidization chamber, e.g., film coating (e.g., by films and/or layers of coating materials, usually spayed into the fluidization chamber), granulation, mixing of different cohesive powders, and/or reactive or non-reactive fluidization-based treatments or unit operations.

The disclosed dry coating systems and methods are environmentally benign, yet effectively convert cohesive powder systems into non-cohesive powder system that can be easily fluidized. Indeed, prior to the disclosed systems and methods, particles below about 30 microns cannot be fluidized in conventional fluidized beds without applying additional external forces, and, typically, particles smaller than about 50 microns cannot be film coated using a fluidized bed process. However, according to exemplary applications of the disclosed systems/methods, micron particles at least as small as 5 microns may be effectively/reliably fluidized using conventional fluidization equipment and, once fluidized, such particles can be subjected to fluidization-based processing, e.g., particle coating techniques and other applications. Accordingly, the systems and methods of the present disclosure permit production of uniformly coated particles at least as small as 5 microns. In implementations of the disclosed systems/methods wherein dry coated particles are fed to a rotating fluidized bed and a centrifugal force is applied/introduced, it has been found that processing of particles down to 1 micron may be achieved with ease.

The disclosed systems and methods offer numerous advantages, as will be readily apparent to persons skilled in the art. For example, the disclosed systems and methods may be employed with conventional fluidization equipment, i.e., there is no need to replace existing capital equipment. Moreover, the disclosed systems and methods can be utilized in a vast array of industrial/commercial applications, including processes involving pharmaceuticals, foods, cosmetics, agrochemicals, defense applications and electronics materials. In addition, as noted above, the systems and methods of the present disclosure further facilitate downstream coating, granulation, mixing, and other reactive and non-reactive fluidization-based processes.

The dry coating step of the present disclosure is to be specifically differentiated from “dry blending” techniques that are disclosed in background art, e.g., U.S. Pat. No. 6,833,185 to Zhu et al. In the dry blending technique of the Zhu '185 patent, the smaller particles (“fluidization additives”) are merely blended into a cohesive powder system. The Zhu '185 patent fails to recognize the advantages associated with the systems and methods of the present disclosure, wherein fine, nano-particles are robustly coated onto the particles of the underlying cohesive powder, thereby ensuring a substantially homogenous and stable powder system for downstream processing, e.g., fluidization. Moreover, in contrast to the dry coating techniques of the disclosed systems and methods, the “dry blending” technique of the Zhu '185 patent is not effective in transforming cohesive powder systems to non-cohesive powder systems at the smaller particle size levels of the present disclosure, e.g., down to 5 microns. Also, in the present disclosure it is demonstrated that guest particles with a larger packing density (“apparent” density) than the cohesive particles are also effective in improving the flow and fluidization behavior of the cohesive host particles.

Additional advantageous features and functions associated with the disclosed systems and methods will be apparent from the detailed description which follows, particularly when read in conjunction with the appended figures. Such additional advantageous features and functions are expressly encompassed within the scope of the present disclosure.

### BRIEF DESCRIPTION OF FIGURES AND TABLES

To assist those of ordinary skill in the art in making and using the disclosed systems and methods, reference is made to the accompanying figures and tables, wherein:

FIG. 1(a) is a field emission scanning electron microscope (FESEM) image of cornstarch particles associated with an exemplary implementation of the present disclosure;

FIG. 1(b) is a FESEM image of Aerosil R972 silica associated with an exemplary implementation of the present disclosure;

FIG. 1(c) is a FESEM image of CAB-O-SIL EH-5 silica associated with an exemplary implementation of the present disclosure;

FIG. 1(d) is a FESEM image of OX-50 silica associated with an exemplary implementation of the present disclosure;

FIG. 1(e) is a FESEM image of silica fabricated in a laboratory at New Jersey Institute of Technology associated with an exemplary implementation of the present disclosure;

FIG. 1(f) is a FESEM image of COSMO55 mono-dispersed hydrophilic spherical silica associated with an exemplary implementation of the present disclosure;

FIG. 1(g) is a FESEM image of P-500 silica associated with an exemplary implementation of the present disclosure;

FIG. 2 is a schematic diagram of a magnetic assisted impaction coating (MAIC) apparatus employed in exemplary implementations of the present disclosure;

FIG. 3 is a schematic diagram of a Hybridizer (HB) apparatus employed in exemplary implementations of the present disclosure;

FIG. 4 is a photograph of a V-shaped blender (VB) employed in exemplary implementations of the present disclosure;

FIG. 5 is a plot showing angle of repose (AOR) for a series of experimental runs associated with the present disclosure;

FIGS. 6(a)-6(d) are SEM images of dry coated cornstarch particles in connection with experimental studies associated with the present disclosure;

FIGS. 7(a)-7(f) are SEM images of dry coated silica/cornstarch systems associated with experimental studies conducted according to the present disclosure;

FIGS. 8(a)-8(d) are additional SEM images of dry coated silica/cornstarch systems (COSMO55 silica) associated with experimental studies conducted according to the present disclosure;

FIGS. 9(a) and 9(b) are schematic diagrams of particle interactions/contacts in guest/host particle systems;

FIG. 10 is a plot of angle of repose (AOR) for dry coated host cornstarch treated for ten (10) minutes in MAIC with different forms of silica;

FIG. 11 sets forth FESEM images of 10-min MAIC coated samples with different silica: (a) EH-5; (b) COSMO55; and (c) P-500.

FIG. 12 sets forth plots of particle size distribution of P-500 silica.

FIG. 13 is a plot of angle of repose (AOR) of 10-min MAIC coated samples with different loading levels of EH-5 silica;

FIG. 14 sets forth FESEM images of 10-min MAIC coated samples with different levels of loading of EH-5 silica: (a) 0.01%; (b) 0.1%; and (c) 1.0%;

FIG. 15 sets forth plots of angle of repose (AOR) of EH-5 silica (1.0 wt %) and R972 silica (1.0 wt %) coated samples using MAIC for 5, 10, 20 and 40 minutes;

FIG. 16 sets forth plots of angle of repose (AOR) of EH-5 silica coated samples using V-blender for 2, 5, 10, 20 and 40 minutes: (a) EH-5 @ 0.1 wt %; and (b) EH-5 @ 1.0 wt %;

FIG. 17 is a plot of the Geldart Fluidization Groups;

FIG. 18 is a plot of fluidization performance of a cornstarch host powder that has been dry coated with R972 silica at 0.01 wt % using MAIC for 10 minutes;

FIGS. 19 and 20 are plots of fluidization performance of a cornstarch host powder that has been dry coated with R972 silica at 0.025 wt % using MAIC for 10 minutes;

FIGS. 21 and 22 are plots of fluidization performance of a cornstarch host powder that has been dry coated with R972 silica at 0.04 wt % using MAIC for 10 minutes;

FIGS. 23 and 24 are plots of fluidization performance of a cornstarch host powder that has been dry coated with R972 silica at 0.05 wt % using MAIC for 10 minutes;

FIGS. 25 and 26 are plots of fluidization performance of a cornstarch host powder that has been dry coated with R972 silica at 0.1 wt % using MAIC for 10 minutes;

FIG. 27 provides SEM images at different magnifications showing a 1.0 wt % coating of hydroxypropyl cellulose (HPC) on a cornstarch host powder that was dry coated with nanosized silica according to the present disclosure;

FIG. 28 provides SEM images at different magnifications showing a 2.0 wt % coating of hydroxypropyl cellulose (HPC) on a cornstarch host powder that was dry coated with nanosized silica according to the present disclosure;

FIG. 29 provides SEM images at 200× and 100× magnification of dry coated cornstarch particles that are granulated according to the present disclosure;

FIG. 30 provides SEM images at various magnifications showing dry coated cornstarch particles that are granulated according to the present disclosure;

FIG. 31 is a plot of fluidization performance for a cornstarch system that is dry coated with 5% nickel according to the present disclosure;

FIG. 32 is a plot of fluidization performance for a cornstarch system that is dry coated with 1.5% TiO₂ (R104) according to the present disclosure; and

FIG. 33 is a plot of fluidization performance for a control system that includes cornstarch dry blended with 1.5% TiO₂ (R104);

FIGS. 34a-34d are SEM images of: (a) pure cornstarch; (b) 0.5 wt % fumed silica (EH-5) dry coated cornstarch; (c) granulated, dry coated cornstarch×500; and (d) granulated, dry coated cornstarch×20 KX;

FIGS. 35a-35d are size distribution plots for cornstarch granules at different granulation conditions according to an exemplary embodiment of the present disclosure;

FIGS. 36a-36d are SEM micrographs of (a) raw aluminum; (b) 0.5 wt % EH-5 coated aluminum; (c) coated aluminum (×500); and (d) coated aluminum (×20 KX), according to an exemplary embodiment of the present disclosure;

FIGS. 37a-37d are plots of particle size distribution of coated aluminum for different operating parameters according to an exemplary embodiment of the present disclosure; and

FIG. 38a-38f are SEM micrographs of coated aluminum particles with R972 silica, as follows: (a) 3-4.5 μm with magnification of 50,000; (b) 3-4.5 μm with magnification of 10,000; (c) 4.5-7 μm with magnification of 50,000; (d) 4.5-7 μm with magnification of 10,000; (e) 10-14 μm with magnification of 50,000; and (f) 10-14 μm with magnification of 10,000;

Table 1 sets forth physical properties of exemplary host particles (cornstarch) and guest particles (silica);

Table 2 sets forth surface coverage of a host powder (cornstarch) with 0.1 wt % EH-5 silica using various processing techniques (MAIC, Hybridizer, V-blender and hand mixing);

Table 3 sets forth surface coverage of a host powder (cornstarch) coated with EH-5 silica at different guest loading levels using MAIC for 10 minutes;

Table 4 sets forth experimental data associated with granulation of cornstarch according to an exemplary embodiment of the present disclosure; and

Table 5 sets forth experimental data associated with experimental processing of aluminum particles according to an exemplary embodiment of the present disclosure.

### DESCRIPTION OF EXEMPLARY EMBODIMENT(S)

The systems and methods of the present disclosure advantageously reduce cohesive forces associated with cohesive powders through a robust dry coating technique, whereby fine, nanosized particles are dry coated onto the particles associated with a cohesive powder system. The fine, nanosized particles are generally uniformly dispersed over the surface of the cohesive powders, and generally define a very sparse layer thereon. Typically, the fine, nanosized particles are coated onto the cohesive powders at levels of about 0.04 to about 2 wt. %. Despite the relatively limited amount of fine, nanosized particles coated onto the particles of the cohesive powder system, the cohesive forces associated with the cohesive powder are generally reduced by at least an order of magnitude or better.

The disclosed fine, nano-particles may take the form of organic or inorganic materials or combinations thereof. Exemplary inorganic materials include: fumed silica, fumed alumina, zeolite, perlite, vermiculite, mica, fumed titanium dioxide, graphite black, carbon black, magnesium oxide and boron nitride. Exemplary organic materials include polyesters, polyurethanes, epoxies, acrylics, polyamides, polyolefins, vinyls and poly(vinylidene fluoride). The cohesive powders to which the fine, nanosized particles may be coated may take a variety of forms, depending on the application of interest, e.g., pharmaceuticals, foods, cosmetics, fertilizers, pesticides, agrochemicals, energetic materials, and the like. Downstream processing, e.g., fluidization, granulation, coating and the like, is facilitated through the disclosed dry coating process, thereby greatly enhancing the processing ease and effectiveness associated with cohesive powder-related processing techniques. To further illustrate applications and implementations of the disclosed systems and methods, attention is turned to experimental testing related thereto.

Powders

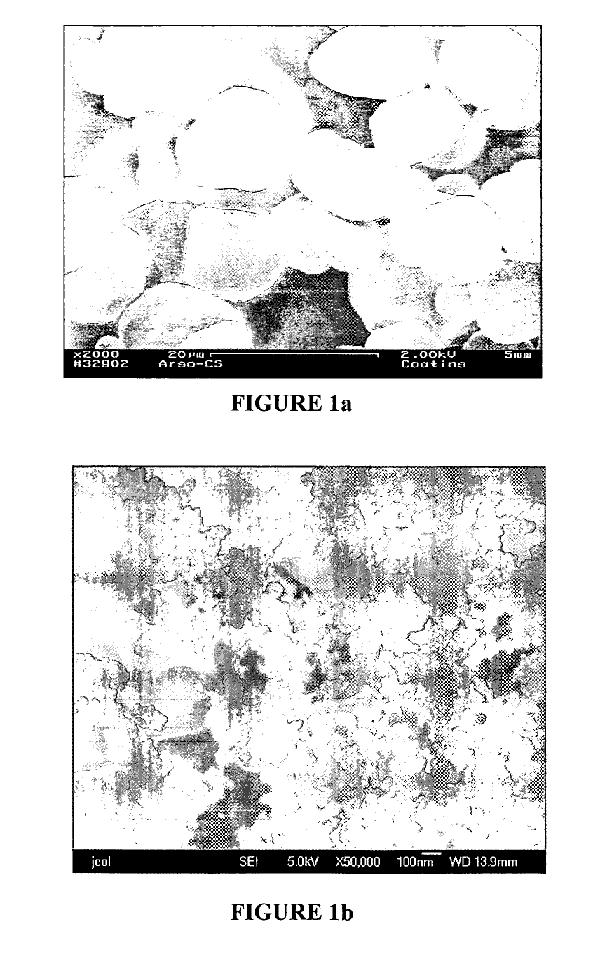

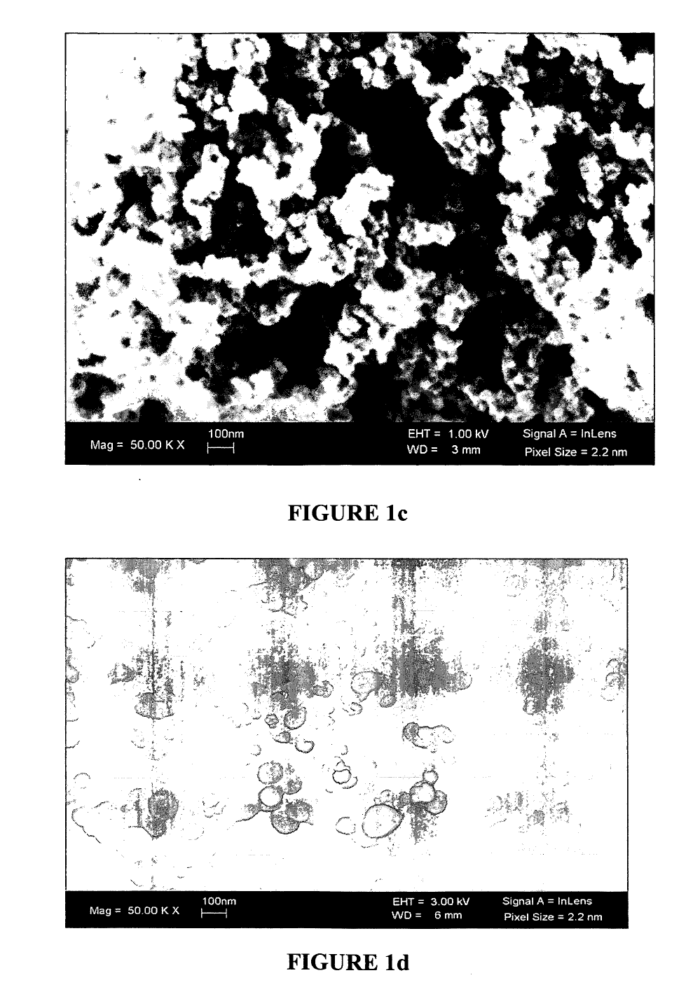

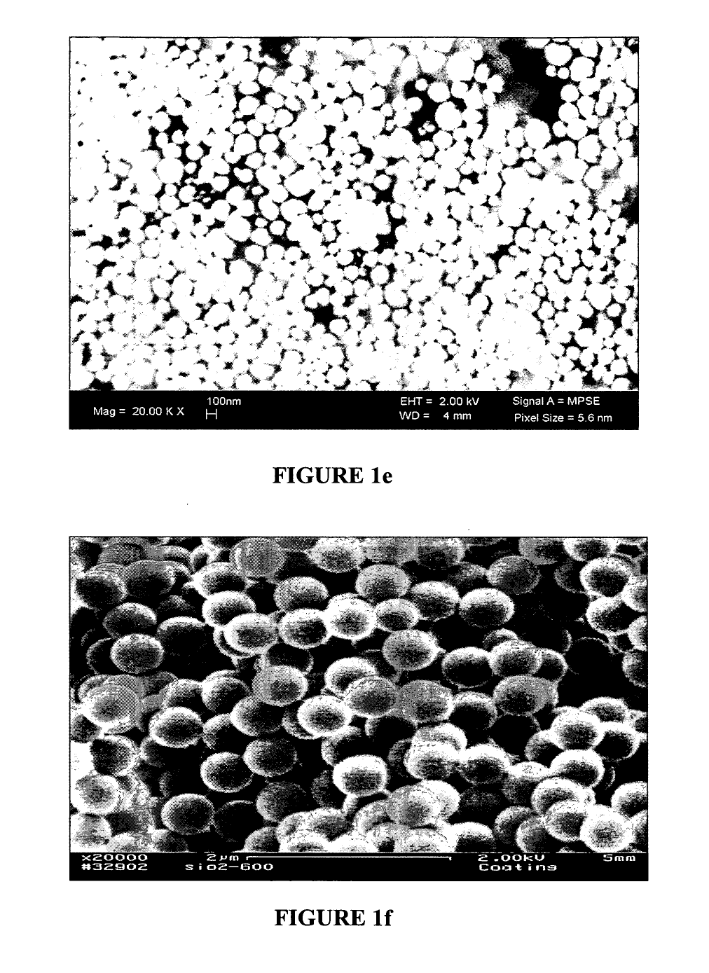

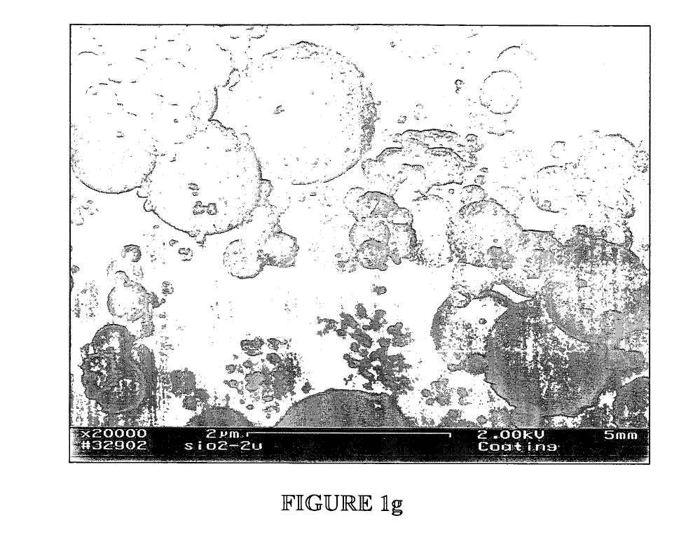

In an illustrative implementation of the disclosed systems and methods, cornstarch (Argo) was used as host particles (i.e., cohesive powder system) for the disclosed dry coating technique. As shown in FIG. 1(a), the field emission scanning electron microscope (FESEM) image indicates that the cornstarch particles were rounded individual particles with a mean size of around 15 microns, which was also verified using a Coulter particle size analyzer. The density of cornstarch was around 1550 kg/m³.

Six different silica particles were used as guest particles (i.e., fine, nanosized particles for coating onto particles associated with the cohesive powder system):

(1) Aerosil R972 silica supplied by Degussa with a specific surface area of 114 m²/g. A FESEM image (FIG. 1(b)) shows highly agglomerated particles with a primary particle size around 20 nm in a chainlike structure. Its surface was modified with dichloryldimethenanolsilane to make it hydrophobic.

(2) CAB-O-SIL EH-5 silica supplied by Cabot (primary particle size also around 20 nm; see FIG. 1(c)), was similar in structure to R972 except that its surface was hydrophilic.

(3) OX-50 silica supplied by Degussa had an average size of about 40 nm (FIG. 1(d)) and was hydrophilic.

(4) 100 nm silica, synthesized in a laboratory at New Jersey Institute of Technology using the Stober process; the synthesized silica was hydrophilic (FIG. 1(e)).

(5) COSMO55 supplied by Catalyst & Chemical Ind. Co. Ltd (Japan) was 500 nm mono-dispersed hydrophilic spherical silica particles (FIG. 1(f)), but tended to form large agglomerates (which needed to be dispersed during the dry coating process).

(6) P-500 hydrophilic silica, also supplied by Catalyst & Chemical Ind. Co. Ltd (Japan), had an average size of around 2.25 microns (FIG. 1(g)) with a wide size distribution.

The particle density of all tested silicas was 2650 kg/m³. The properties, size, and other physical properties of all particles (i.e., host and guest particles) used in this set of experiments are summarized in Table 1 appended hereto.

Dry Coating Processes

The percentage by mass of guest particles used in the dry coating experiments described herein was calculated based on a target of 100% surface coverage of the host particles with a monolayer of guest particles. It was assumed that all guest particles were de-agglomerated and of the same size, that both host and guest particles were spherical, and that the host and guest particles would not deform during the dry coating process. Based on these assumptions, the weight percentage of guest particles for 100% surface coverage was calculated as:

$$\begin{matrix}Gwt \% = \frac{( {Nd}^{3} {ρ}_{d} )}{( {D}^{3} {ρ}_{D} ) + ( {Nd}^{3} {ρ}_{d} )} \times 100 \\ Here \text{:} & ( 1 ) \\ N = \frac{4 {( D + d )}^{2}}{{d}^{2}} & ( 2 )\end{matrix}$$

From Equation (1), the weight percentages of guest particles needed to coat 15 μm cornstarch particles are 0.91%, 19.6% and 57.6%, respectively for 20 nm, 500 nm and 2.25 μm silica. Accordingly, in the experiments described herein, 1.0 wt % of nanosized silica and 20 wt % 500 nm silica were used. In addition, dry coating experiments were also performed with 0.1 wt % and 0.01 wt % of the nano silica and 2.0 wt % of the 500 nm silica. For the large 2.25 μm silica guest particles, experiments were only conducted using 2.0 wt %, even though this level is much less than theoretically needed to produce a monolayer of guest particles on the surface of the cornstarch.

Two different dry coating devices and a dry mixer were studied to determine the coating performance as described below:

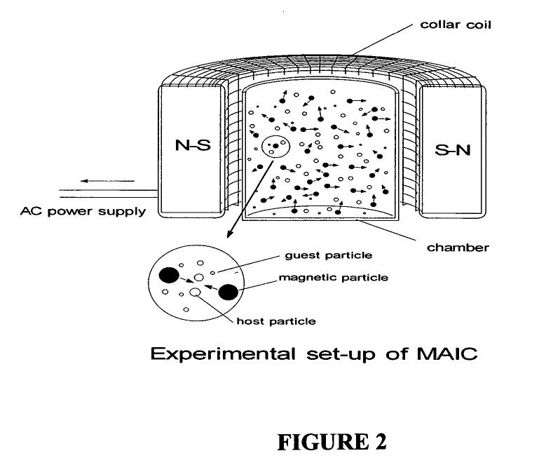

(1) Magnetic Assisted Impaction Coating (MAIC) apparatus: FIG. 2 is a schematic diagram of a MAIC apparatus. The oscillating magnetic field generated by the coil is used to accelerate and spin the large magnetic particles mixed with the host and guest particles, promoting collisions between the particles and with the walls of the vessel. Since the magnetic particles appear to “fluidize” the host and guest powders (even though no fluidizing gas was used), “soft” coating occurs by powder impaction. The magnetic particles were barium ferrite particles coated with polyurethane and had a size range from 1.4 to 1.7 mm. The weight ratio of magnets to host and guest particles was 3 to 1. Unless stated otherwise, the processing time using the MAIC device was ten (10) minutes.

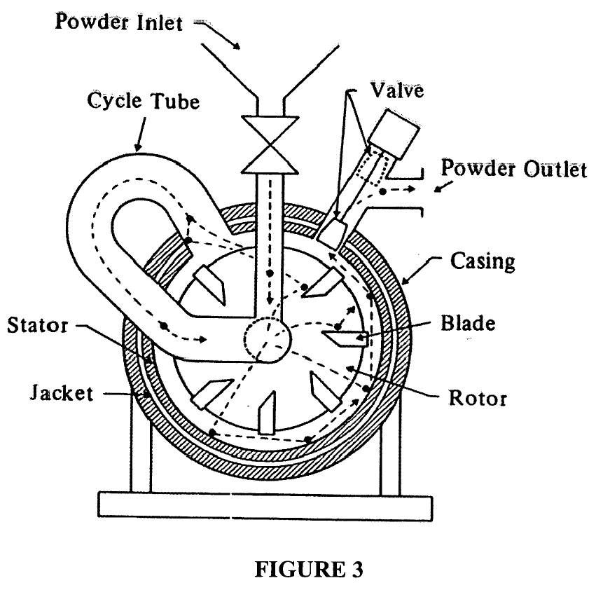

(2) Hybridizer (HB) coating apparatus: FIG. 3 is a schematic diagram of a Hybridizer apparatus. It consists of a very high-speed rotating rotor with six blades, a stator and a powder re-circulation circuit. The rotor diameter was 118 mm, and the outer edge of each blade was 35 nm from the rotational axis. The powder mixture (host and guest particles) was subjected to high impaction and dispersion due to collisions with the blades and the walls of the HB device and continuously re-circulated in the machine through the cycle tube. Particle coating was achieved due to the embedding or filming of the guest particles onto the host particles by high impaction forces and friction heat. Since the HB device operates at very high rotating speed, a very short processing time was required to achieve the desired dry coating of the host particles. The operating conditions of the HB device were 6000 rpm for 2 minutes.

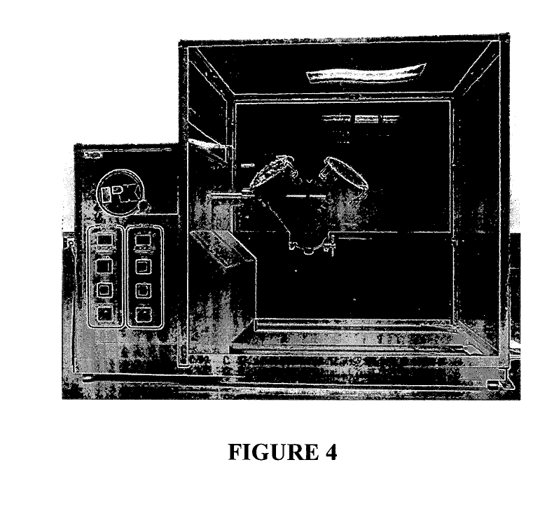

(3) V-shaped blender (VB) mixing/coating: FIG. 4 is a photograph of a V-shaped blender (Patterson-Kelly—BlendMaster) employed in the present experimental study. The blender achieved good powder mixing as the vessel was rotated slowly and, during each rotation, the powder flowed into the two arms followed by powder pouring back towards the apex of the system. In the present study, the blender was operated at 25 rpm and an internal stirring bar (also called an agitator bar) that rotates at very high speed (3600 rpm) was used to enhance the mixing behavior inside the chamber. The tips of the intensifier bar extended 55 mm from the rotational axis. For each batch, 150 grams of particles were charged into a 4 quart vessel and processed for 2 to 40 minutes. A small amount of primary (host) powder was first mixed with the guest material in a plastic zip-lock bag, and then this mixture was added to the rest of the primary powder in the vessel of the V-blender. Unless stated otherwise, the processing time in the V-blender was 10 minutes.

(4) Hand mixing: Experiments using simple hand mixing were also conducted as a control. In this control procedure, the primary material was placed in a bottle along with the guest material and then the sealed bottle was shaken by hand for approximately 10 minutes.

Characterization

A Coulter LS 230 particle size analyzer and a LEO 1530 VP field emission scanning electron microscope (FESEM) were used to measure the particle size distribution of the host and guest particles. A Hosokawa powder tester (PT-N) was used to measure the angle of repose (AOR) of the dry coated cornstarch particles to characterize their flowability. The procedure used to measure AOR was as per ASTM standard (ASTM D6393-99, “Bulk Solids Characterization by CARR Indices”), and each reported reading was an average of at least three (3) observations. FESEM images were also used to observe the distribution of the guest particles on the surface of the host particles and MATLAB was used for image analysis.

Experimental Results and Discussion

A. Evaluation of Dry Coating Processes

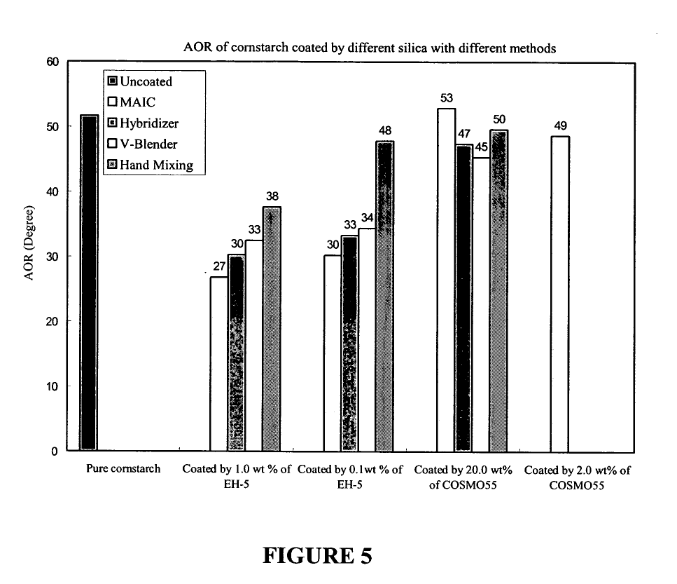

Coating experiments were performed using the same amount of silica guest particles with cornstarch as the host particles in the three different devices. Hand mixing was used as a control. The AOR of the coated products (which is a measure of flowability) was plotted in FIG. 5 to compare the coating efficiency of different coating devices. For a 1.0 wt % coating of EH-5, which corresponds to a 100% monolayer surface coverage, the plot shows that MAIC, HB and VB are all capable of significantly improving the flowability of cornstarch and reducing its AOR from 52 degrees to 26, 30 and 33 degrees, respectively. Even hand mixing reduces the AOR to 38 degrees.

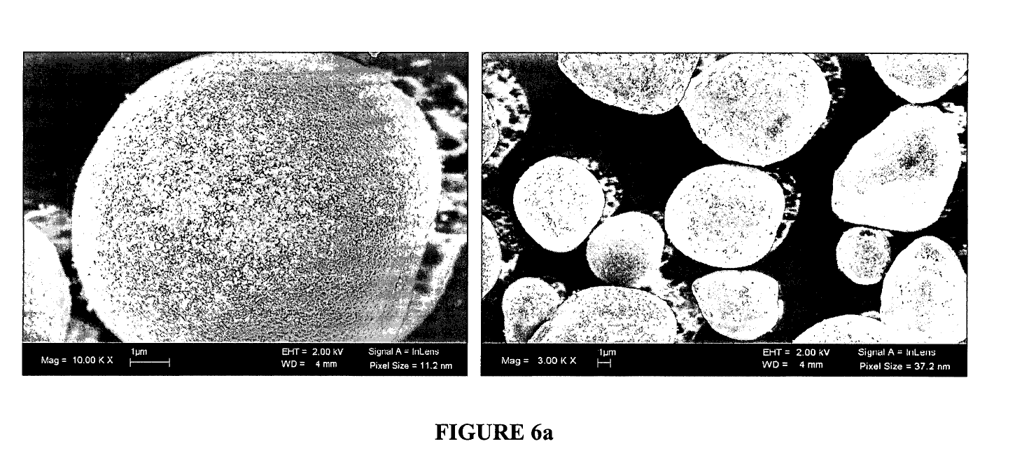

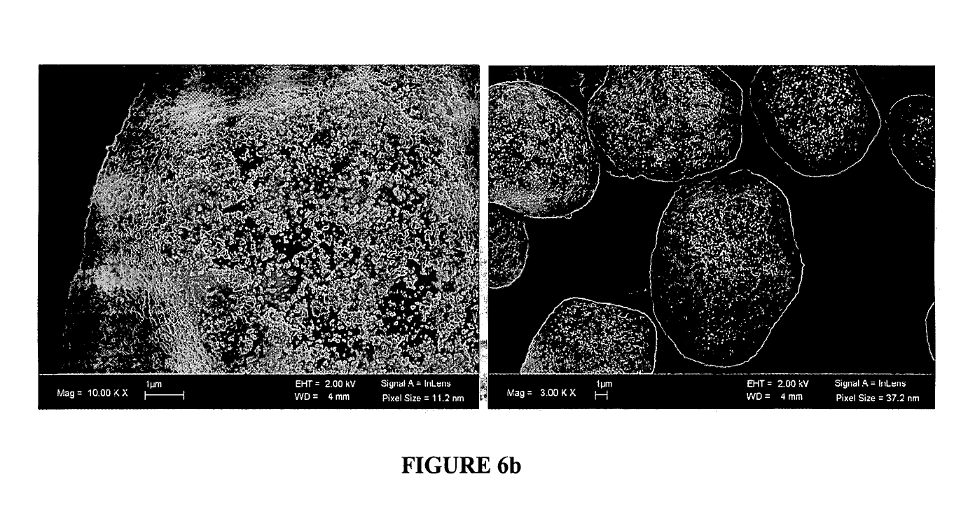

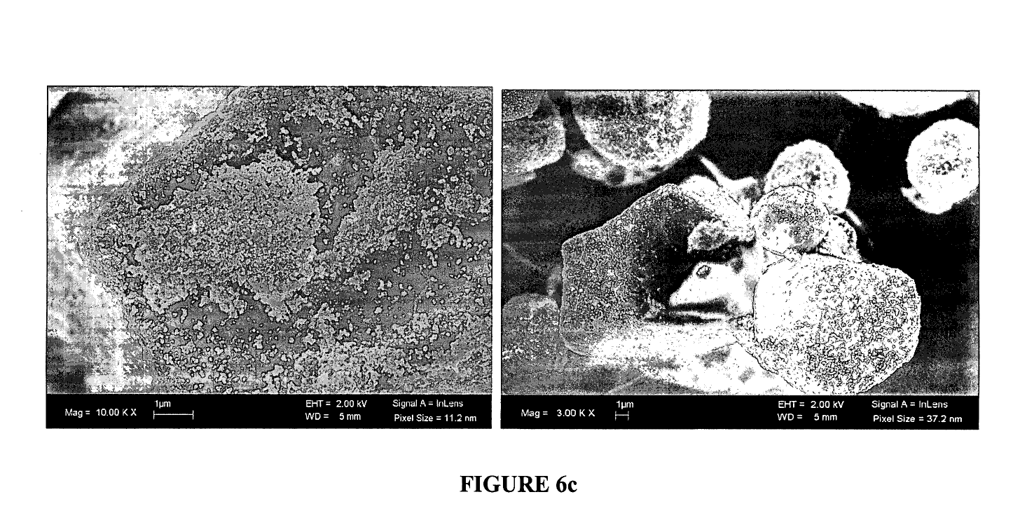

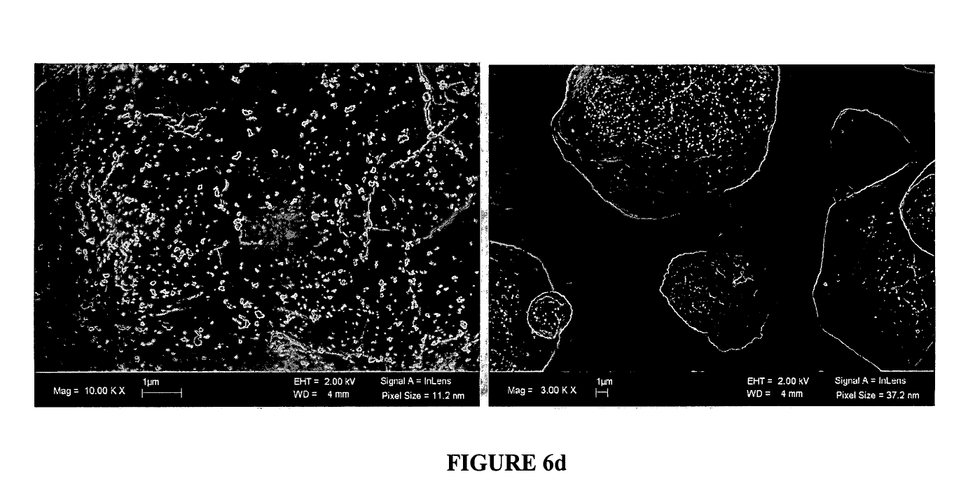

The corresponding SEM images are shown in FIGS. 6(a)-6(d). For MAIC and HB coated products (FIG. 6(a) and FIG. 6(b)), the fumed silica particles, EH-5, are dispersed evenly onto the cornstarch particles and there are no observed large silica agglomerates. The V-blender was also shown to be capable of coating fumed silica onto cornstarch, but the distribution of the fumed silica on the surface of the cornstarch particles was not uniform. As shown at the left of FIG. 6(c), agglomerates of fumed silica look like “patches” on the cornstarch surface and some small silica agglomerates, as well as uncoated cornstarch surfaces are also observed (see FIG. 6(c) at right). These results indicate that the de-agglomeration efficiency of the V-blender was not as good as MAIC and HB at the tested operational conditions.

A reasonable explanation for this behavior is that in the MAIC device, the small magnets spin and rotate very fast, leading to many repeated collisions of the cornstarch particles with each other, with magnets, and with the vessel walls, helping de-agglomeration of the EH-5 silica particles so as to obtain a “smooth” coating surface. For the Hybridizer, due to its very high-speed rotation, the impaction and dispersion forces between particles are very strong, resulting in a uniform coating. Compared to these devices, the V-blender, even with an internal stirring bar, is not capable of fully dispersing the guest particles and, hence, agglomerated particles are found on the surface in the form of “patches.” Hand mixing is only capable of coating part of cornstarch surface, as shown at the left of FIG. 6(d), and large individual agglomerates of EH-5 silica are observed on the coated product, as shown at the right of FIG. 6(d) (see circled area).

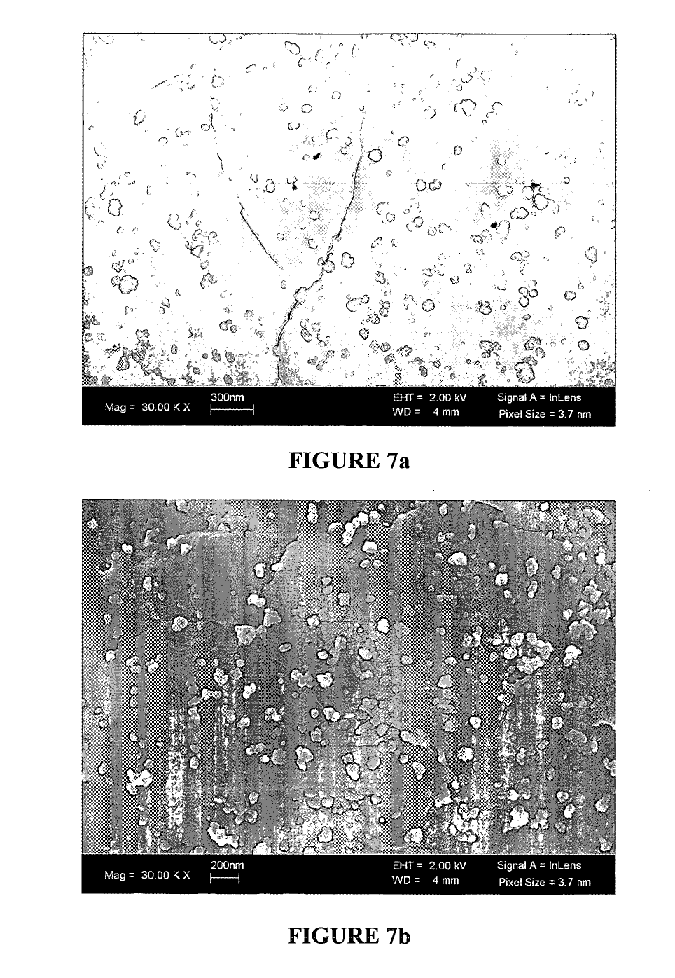

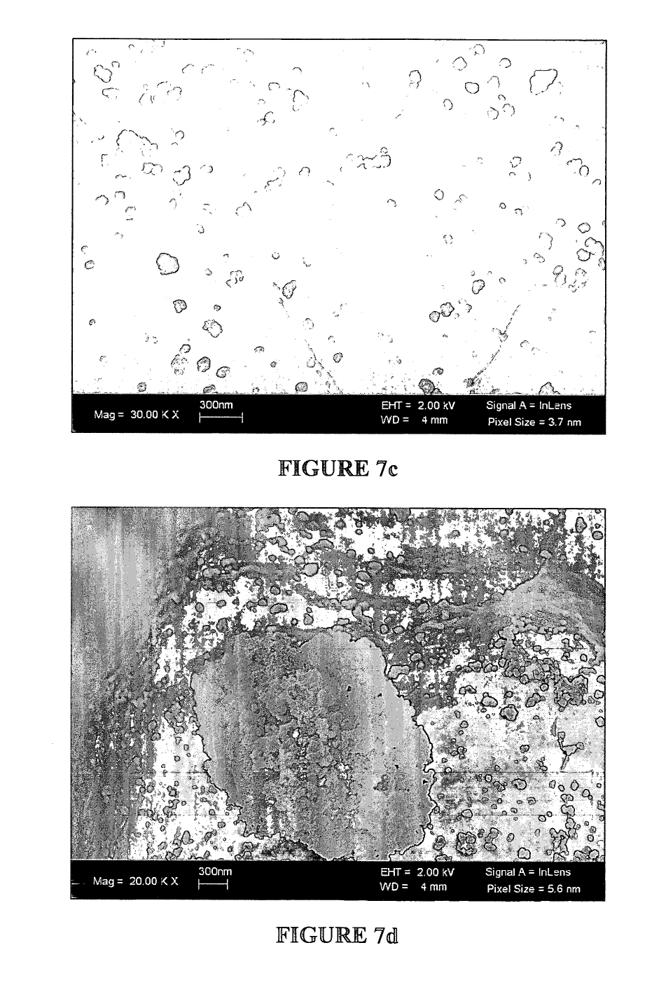

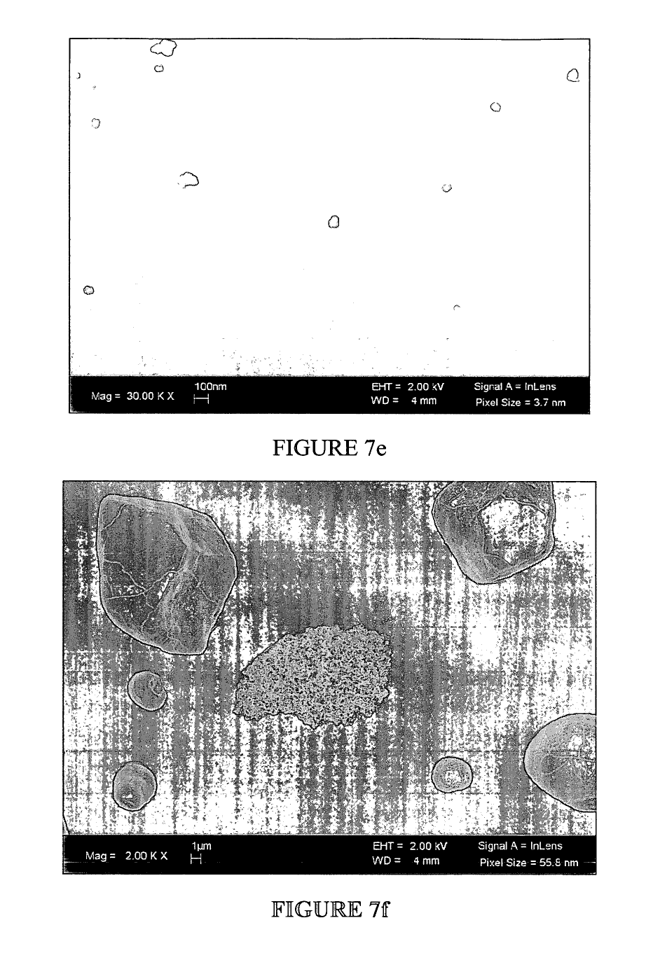

Nonetheless, these tests confirmed that nanosized fumed silica are effective flow agents for cohesive cornstarch particles, and the flowability of the three machine processed samples was superior to the hand mixed sample. For a 0.1 wt % coating of EH-5, which corresponds to a theoretical 10.9% surface coverage, FIG. 5 shows that dry coating using the MAIC, HB and dry blending using the V-blender equipment functioned to improve the flowability of the cohesive cornstarch and reduce the AOR to 30, 33 and 34 degrees, respectively. However, for this small amount of silica addition, hand mixing had no favorable effect on the flowability of the cohesive cornstarch. Corresponding SEM images clearly show that the nanosized guest particles are uniformly coated on the surface of the cornstarch for both the MAIC and HB processed samples, as shown in FIG. 7(a) and FIG. 7(b). As stated before, the V-blender was less effective in de-agglomeration of EH-5 silica agglomerates, thus less guest particles were observed on the surface of the VB processed cornstarch particles (FIG. 7(c)) and some small agglomerates were also observed, as seen in FIG. 7(d). For hand mixing, only a few guest silica particles were attached to the cornstarch surface, as seen in FIG. 7(e), and large silica agglomerates were detected (see FIG. 7(f)).

These test results demonstrate that both MAIC and HB systems are capable of deagglomerating and dispersing nanosized guest particles. Moreover, the MAIC and HB systems were effective for dry coating the nanosized guest particles evenly on the surface of cornstarch, a cohesive host powder. Compared to the MAIC and HB devices, the V-blender was less effective in breaking down the small agglomerates of fumed silica under the operational conditions tested; thus, the coating efficiency of the V-blender was lower than the MAIC and HB devices. For hand mixing, the impaction forces between the particles were not sufficient to break down the silica agglomerates; hence, very little coating of the host particles occurred.

A quantitative evaluation of the deagglomeration/dispersion capabilities of the different devices was also conducted by image analysis of the SEM images of FIGS. 7(a), 7(b), 7(c) and 7(e), using MATLAB to calculate the surface coverage of the guest particles. The results, shown in Table 2 (appended hereto), indicate that a surface coverage of 1.72%, 6.74%, 13.2% and 10.72% were obtained for cornstarch processed by hand mixing, VB, HB and MAIC, respectively. As stated previously, the theoretical surface coverage for a system that includes a 0.1 wt % coating of fumed silica is 10.9%, which is very close to the experimental results achieved using the HB and MAIC devices in processing a cornstarch/silica system, as described herein. While the coverage by the V-blender was a reasonable 6.74%, such coverage includes small agglomerates and, hence, dispersion of the guest particles was not even. These results show that both the HB and MAIC devices can effectively deagglomerate 0.1 wt % of silica particles and dry coat them evenly onto the cornstarch surface. The V-blender was not nearly as effective at the tested conditions and the coating was not nearly as robust since it consisted of many small agglomerates. Effective levels of dry coating were not achieved by hand mixing, as described herein.

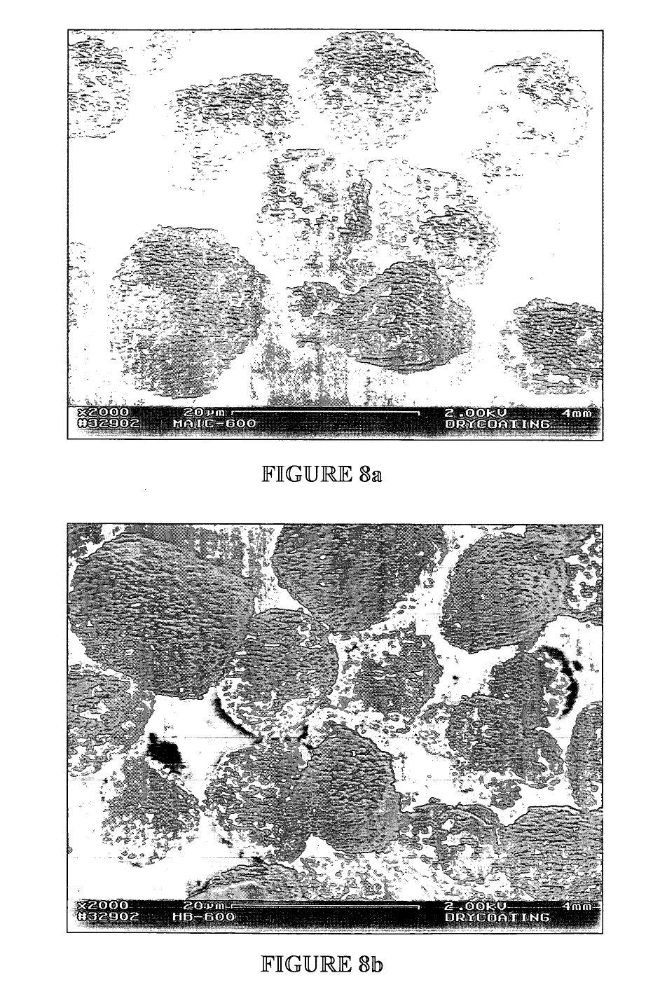

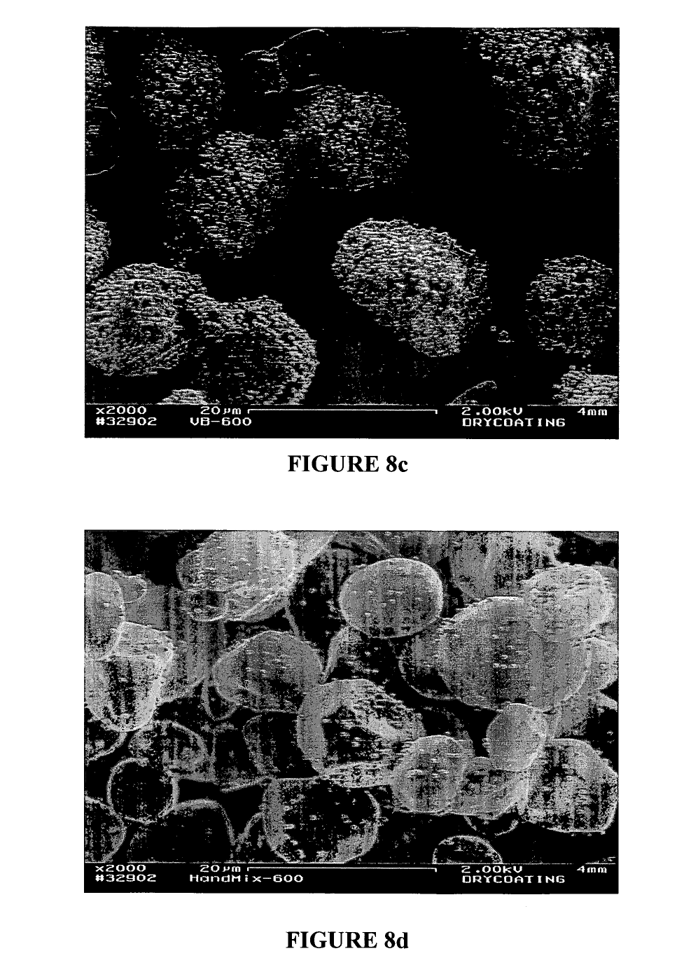

Similar experiments were also conducted with 500 nm silica particles, as shown in FIG. 5. The AOR results for coating tests using 20.0 wt % of COSMO55 silica, corresponding to approximately 100% surface coverage, were 53, 47, 45 and 50 degrees for MAIC, HB VB and hand mixing, respectively. Although the AOR results indicate that the 500 nm guest silica particles had no apparent effect on the flowability of cornstarch, the SEM images indicate that a reasonable coating of guest particles occurred using MAIC, HB and VB, as shown in FIGS. 8(a), 8(b) and 8(c). For hand mixing, however, only a few particles are seen on the surface of the cornstarch, as shown in FIG. 8(d). These results indicate that all three tested devices are capable of deagglomerating the larger 500 nm silica particles and dry coating them onto the surface of cornstarch; however, coating could not be achieved with the COSMO55 silica by hand mixing.

Coating experiments with 2.0 wt % of COSMO55 silica particles were also conducted using the MAIC device. The AOR of the coated sample was 49 degrees, which indicates that dry coating of such guest particles onto the cornstarch host powder had no significant effect on the flowability of the cornstarch.

B. The Effect of Guest Particle Size

The experimental results set forth above demonstrate that a large improvement in the flowability of cornstarch particles (i.e., cohesive host powder) is obtained by dry coating the cornstarch particles with nanosized silica (i.e., nanosized guest particles), but that dry coating the cornstarch particles with 500 nm (i.e., non-nanosized) guest particles fails to improve the flowability of a cohesive host powder (see test results reflected in FIG. 5). These results demonstrate that the size of the guest particles plays an important role in the effectiveness of the dry coating step for purposes of downstream processing of cohesive powders, and that the dry coating of nanosized guest particles onto cohesive host particles is effective in enhancing the downstream properties of the underlying cohesive powders, whereas dry coating of non-nanosized is ineffective therefor.

(i) Theoretical Confirmation of Particle Size Effect

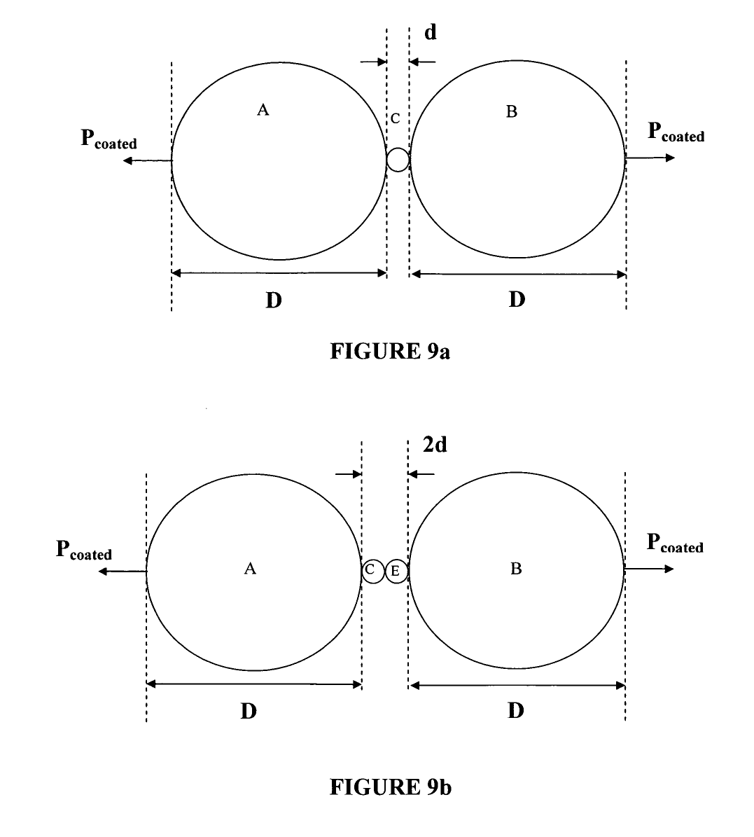

At least an order of magnitude effect difference in cohesive forces between two host particles can be estimated based on reductions in van der Waals forces associated with interpositioning of a guest particle. Two coated particles can have either a single contact or multiple mutual contacts, assuming the coated particles are sparsely (but sufficiently) coated by guest particles. For the sake of providing an order of magnitude analysis, however, a single contact between a guest particle of one host particle to another host particle is considered (see FIG. 9(a)). However, it is noted that there are also contacts between two guest particles on two different hosts (shown in FIG. 9(b)) that can occur.

For the case schematically depicted in FIG. 9(a), it is assumed that the guest particle (C) is attached to the host particle (A), on the left, and is in contact with the host particle, B, on the right. In dry coating, there is some minor deformation of the host or guest or both. Hence, the van der Waals attraction between A and C is higher than the attraction between C and B, which particles are simply in contact without significant deformation [Ref. 8]. Therefore, the amount of force required to pull them apart, i.e., the coated particle on left (assembly of particles A and C) and the host particle on the right, B, is given by the following equation:

$${P}_{coated} ≅ \frac{A}{12} \frac{ⅆ D}{ⅆ + D} \frac{1}{{h}_{o}^{2}} \qquad ( 3 )$$

Here A is the Hamaker constant for these materials (assumed to be approximately the same), and hₒ is the atomic scale separation between the two, for which a value of 0.165 to 0.4 nm is usually used. The total van der Waals attraction force between B and the assembly of particles A and C involves a second term, because there is also a van der Waals attraction between particle A and particle B having a spacer C between them. That term is given by

$$P \approx \frac{A}{12} [ \frac{D}{2 {( 2 {h}_{o} + d )}^{2}} ] \qquad ( 4 )$$

but is several orders of magnitude smaller than the first term, hence it can be neglected.

Equation (3) is a simplification of the exact equation, given by Rumpf [Ref. 14], which includes two terms. The first term is the right hand side of equation (3), and the second term is the right hand side of equation (4). Rumpf's equation has also been used by Huber and Worth [Ref. 15]. Since d is much smaller than D, equation (3) can be further simplified as

$${P}_{coated} ≅ \frac{A}{12} d \frac{1}{{h}_{o}} \qquad ( 5 )$$

If the two particles are not coated, then the van der Waals attraction force between the two is simply given by,

$${P}_{uncoated} ≅ \frac{A}{12} \frac{D}{2} \frac{1}{{h}_{0}^{2}} \qquad ( 6 )$$

For particles of size of the order of microns, Molerus and Massimalla/Donsi point out that equation (6) gives an unrealistically high value of the attraction force and they suggest that, rather than using the size of the particles in contact, D should represent the asperities of the particles and that a typical value of this parameter is 0.2 μm.

Then the ratio between the force required to detach the coated particle compared to the force to detach an uncoated particle is:

$$\frac{{P}_{coated}}{{P}_{uncoated}} = 2 \frac{d}{D} \qquad ( 7 )$$

where D is either the diameter of the host particle or the size of the asperities ˜0.2 μm depending on the size of the host particles (assuming that the Hamaker constants are approximately the same). However, the guest material can be selected in such a manner as to have reduced values for the Hamaker constant in equation (3).

It is noted that this result is the same as that obtained by Mei et al. [Ref. 10], who analyzed the cohesion force with and without the presence of a guest particle (as illustrated in FIG. 9a) by extending the JKR theory. Mei et al. provide the following equation for the ratio of “adhesion” force between coated and uncoated particles, i.e., the force needed to pull them apart:

$$\frac{{P}_{coated}}{{P}_{uncoated}} = 2 \frac{d}{D} \frac{Δ γ}{Γ} ≅ 2 \frac{d}{D} \qquad ( 8 )$$

When the contact case of FIG. 9b is analyzed, it is clear that the weakest contact is that between the two guests, C and E, which should give the magnitude of the force required to break them apart. Again, the total force between the particles A and B include several terms, i.e., direct attraction between particles C and E, particles A and B, particles A and E, as well as particles B and C. However, the last three terms are several orders of magnitude smaller than the first term (attraction between particles C and E), which can then be used to estimate the breaking force.

$${P}_{coated} ≅ \frac{A}{12} \frac{d}{2} \frac{1}{{h}_{0}^{2}} \qquad ( 9 )$$

Dividing equation (9) by equation (6)

$$\frac{{P}_{coated}}{{P}_{uncoated}} = \frac{d}{D} \qquad ( 10 )$$

which differs from equation (7) only by a factor of 2.

An analysis of multiple contacts yields essentially the same results, i.e., the reduction in the contact force due to the presence of small guest particles is proportional to the size ratio between the guest and host particles (or asperities of the host particles), and for a fixed host size, would only depend on the size of the guest particle. In other words, the reduction in the cohesion force is inversely proportional to guest particle size. Thus, the cohesion force between cornstarch particles in the presence of a 20 nm guest silica particle is much less (about 4%) than the cohesion force in the presence of a 500 nm guest silica particle.

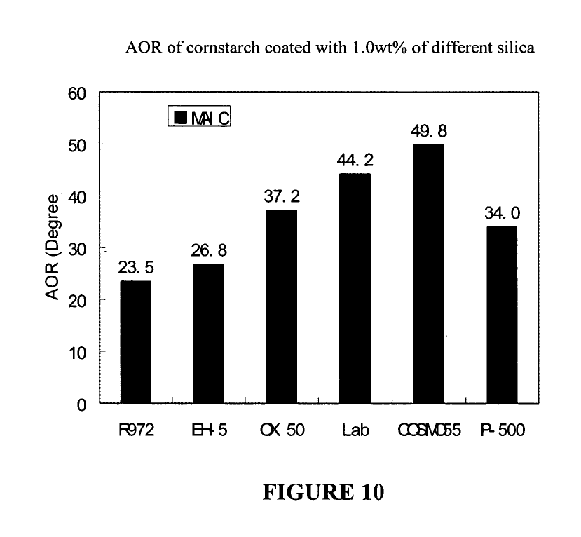

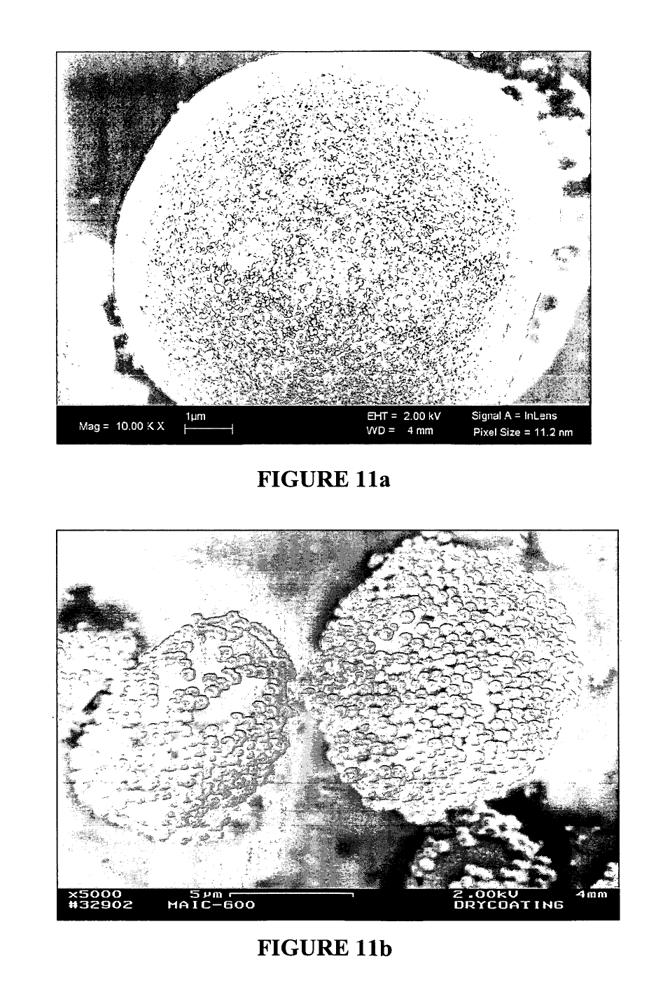

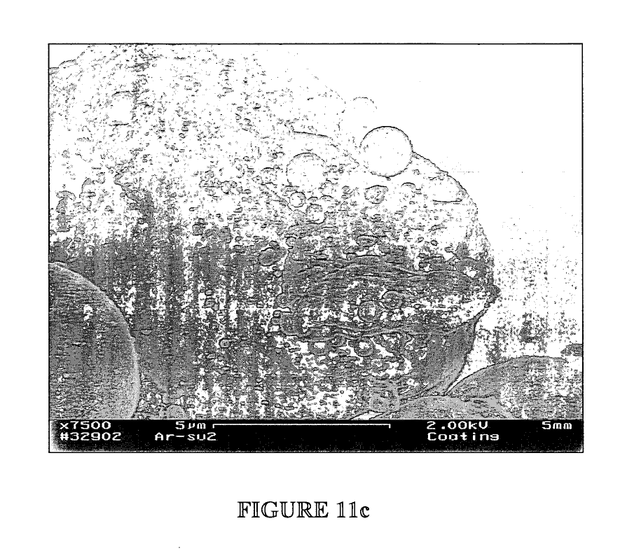

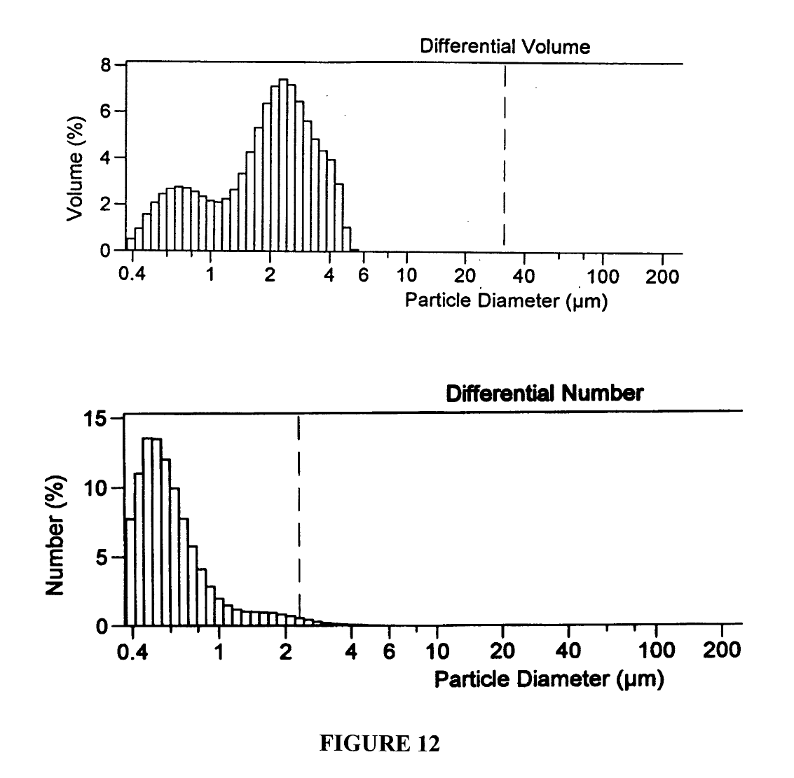

To obtain a better understanding of this phenomenon, 1.0 wt % of R972, OX-50, lab synthesized 100 nm silica and 2.25 μm silica particles were used as guest particles (in addition to the EH-5 and COSMO55 particles that were previously tested) for coating cornstarch with an MAIC device. As shown in FIG. 10, the results indicate that the AOR for the different sized silica particles monotonically decreases as the guest particle size is reduced, except for the 2.25 μm silica. The AOR of the 2.25 μm silica coated cornstarch decreases from 52 to 34 degrees, which contradicts the previously stated conclusion. However, FESEM images (FIG. 11a and FIG. 11b) of the coated cornstarch particles indicate that the 500 nm and 20 nm guest particles appear to be almost mono-dispersed and evenly coated onto the cornstarch particle surface. However, for the 2.25 μm silica guest particles, the particle size analyzer results (as shown in FIG. 12) indicate it has a wide size distribution. The corresponding SEM image of the coated product also confirms that there is a large amount of nanosized silica on the cornstarch surface (FIG. 10c), even though the average guest particle size is 2.25 μm. It is believed that, consistent with the overall conclusions set forth in the present disclosure, these small nanosized particles are responsible for the improvement in the flowability of the coated cornstarch in this experimental study.

C. The Effect of Guest Particle Amount

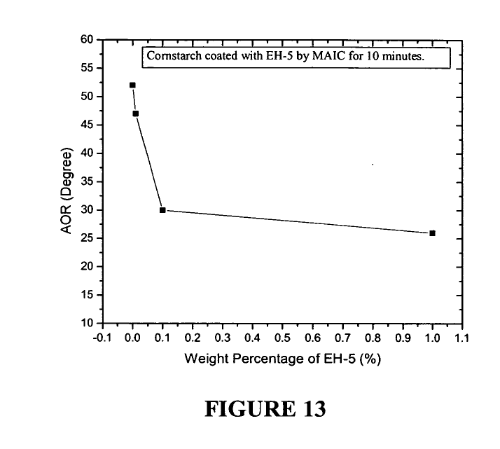

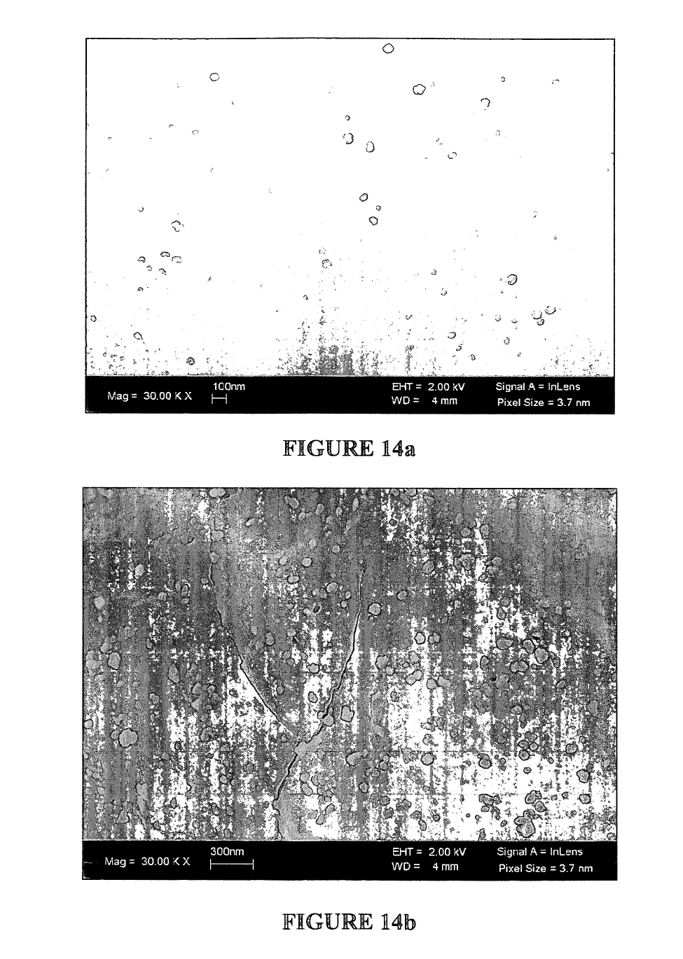

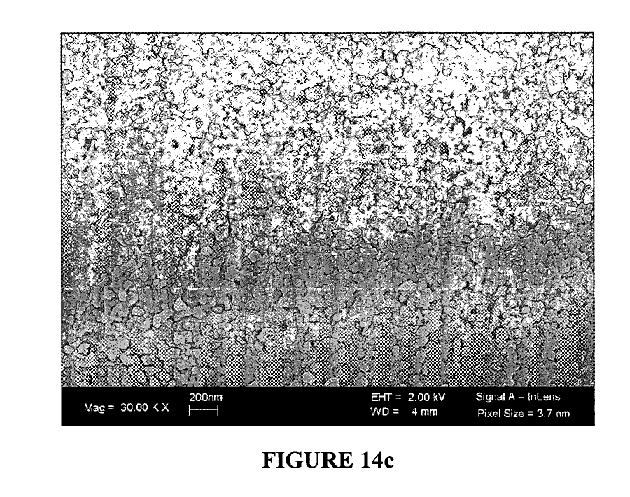

As shown in FIG. 13, the AOR results for the MAIC-coated samples indicate that the flowability of cornstarch is improved by increasing the amount of nanosized particles (up to some limiting amount). By increasing the amount of guest particles (EH-5) from 0 to levels of 0.01, 0.1 and 1.0% by weight, respectively, the corresponding AOR is reduced from 52 to 47, 30 and 27, respectively. FESEM images (FIG. 14) also clearly indicate that the surface coverage of the guest particles increases as the weight percentage of nanoparticles is increased. This is due to a decrease in direct contact of cornstarch particles with one another, which results in a reduction of Van der Waals force between the host particles, as discussed above. When the coating level is very low, such as 0.01%, there is a high probability that no guest particles are present between two cornstarch particles, i.e., the host particles are able to directly contact each other, but as the guest particle level is increased, a greater likelihood exists that guest particle(s) will be present between adjacent host particles, thereby reducing cohesion there between.

Image analysis using MATLAB was also performed for the images shown in FIG. 14. The MATLAB results are set forth in Table 3 and indicate that surface coverages of 1.96, 10.72 and 90.23% are obtained for 0.01, 0.1 and 1.0 wt % silica loading, respectively. It is noted that the experimental surface coverage for the different silica loading is very close to the theoretical calculations, which are 1.1, 10.9 and 100%, respectively. The MATLAB results further indicate that the MAIC system appears to deagglomerate the guest particles more effectively and disperse them evenly onto cornstarch host particles. These results and the corresponding AOR results also suggest that silica levels above 1.0 wt % do not yield further improvements in reduced cohesion.

D. The Effect of Processing Time

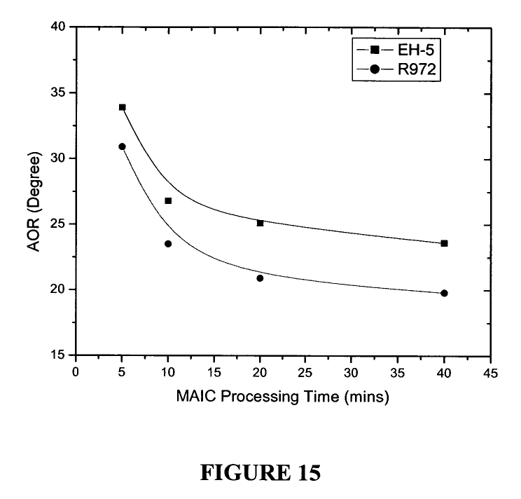

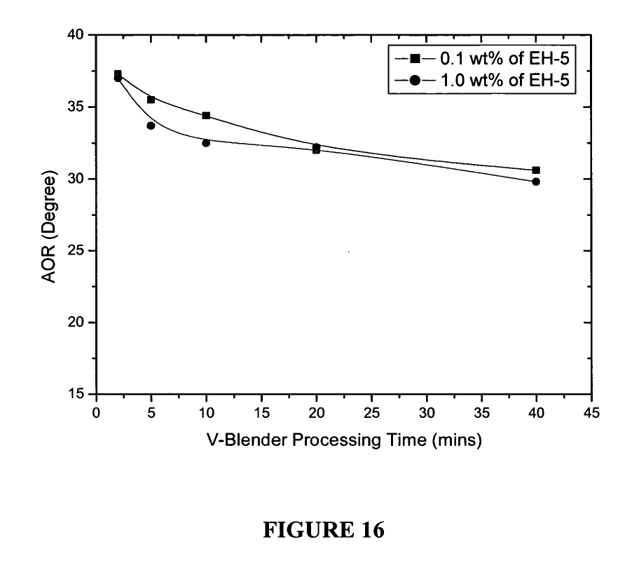

Experiments were also conducted to determine the effect of processing time using MAIC and VB systems. Since HB processing time is generally quite small, i.e., on the order of two (2) minutes, the effect of processing time was not studied in the HB system. FIG. 15 shows the test results for the MAIC system, indicating that when using 1.0% of EH-5 as the coating material, the AOR decreased from 33.9 to 23.6 as the processing time is increased from five (5) to forty (40) minutes. When R972 silica was used as the guest particles, the AOR decreased from 30.9 to 19.8. As shown in FIG. 1, both of these nanosized silica particles are highly agglomerated materials. Since MAIC is a relatively “soft” coating device, a longer processing time favors deagglomeration of the guest particles and a more uniform and larger surface coverage, which in turn results in better flowability. Similarly, as shown in FIG. 16, a longer processing time improves the AOR results for the VB system for both 0.1 and 1.0 wt % of EH-5 silica loading. Thus, longer processing time in a VB system generally improves the deagglomeration and subsequent dispersion of nano-silica agglomerates.

E. The Effect of Hydrophilic/Hydrophobic Surface Properties

The results shown in FIG. 15 also indicate that hydrophobic nanosized silica is more effective in improving the flowability of cornstarch host particles than hydrophilic silica. A hydrophobic silica surface is obtained by treating the hydrophilic silica surface with an appropriate hydrophobic agent, e.g., dimethyldichlorosilane. In the case of dimethyldichlorosilane treatment, an alkyl ended surface molecule results, which is water repellent. The water repellent surface reduces moisture adsorption and the formation of liquid bridges, rendering the cornstarch host particles less cohesive, thereby improving their flowability.

Furthermore, it is known that the surface of cornstarch is rich in hydroxyl groups. Hydrophilic silica also has a surface that contains hydroxyl groups, but hydrophobic silica has a surface that contain alkyl groups. Van der Vegate and Hadziioannou [Ref. 16] have shown that the mean adhesion force (measured by AFM) between hydroxyl groups is 0.9 nN, but is only 0.3 nN between an alkyl group and a hydroxyl group. This implies that the adhesion force for a hydrophobic silica-coated cornstarch will be smaller than the adhesion force for a hydrophilic silica-coated cornstarch (as per the AFM measurements). Thus, as shown in FIG. 15, hydrophobic silica is more effective in improving the flowability of cornstarch as compared to hydrophilic silica.

G. Conclusions

The experimental testing set forth herein demonstrates that it is possible to improve the flowability of a host cohesive powder, e.g., cornstarch, by dry coating the particles that form the cohesive powder with nanosized guest particles, e.g., nanosized silica, using appropriate dry coating techniques. For example, conventional dry coating devices, such as MAIC and HB, may be employed to effect desirable dry coating results. A V-Blender can also be used, but is generally less effective on a comparative basis, especially for very small amounts of guest particles. Although it is possible to obtain a uniform coating of 500 nm silica particles on cornstarch by processing in MAIC and HB systems, the dry coating with such non-nanosized guest particles does not improve the flowability of the cohesive host powder; the large guest particles do not sufficiently reduce the van der Waals forces between the host-particles.

Based on the original Rumpf model for estimating the adhesion force between coated particles, an equation is provided herein that demonstrates that the amount of reduction in the cohesion force for the coated particles is inversely proportional to the size ratio of the guest and the host particles (or for large host particles, the size ratio of the guest particles and the asperities of the host particles), indicating that smaller guest particles provide a larger reduction in the cohesive force. The experimental results agree with this theoretical prediction, with the exception of 2.25 μm P-500, for which, the improvement in flowability observed is attributed to the large amount of fines present.

According to the present disclosure, it has also been demonstrated that increasing the amount of 20 nm silica (within a certain limit) as well as increasing the processing time, when using MAIC as the coating device, improves the flowability of the cohesive powder, i.e., cornstarch. An increase in the processing time makes the coating more even, and reduces the size of very small agglomerates of guest particles adhered on the surface of the host particles. Hence, the “effective” guest particle size is generally reduced as a function of processing time in the dry coating system. Moreover, hydrophobic nanosized silica was demonstrated to be more effective in improving the flowability of cornstarch than hydrophilic nanosized silica due to the elimination of liquid bridges, e.g., if any moisture is present, and the reduction of the adhesion force between the treated guest and host particles.

According to an exemplary implementation of the disclosed dry processing/dry coating methods/techniques, cohesive cornstarch powder is coated with different size silica particles. For nanosized silica guest particles, field emission scanning electron microscope (FESEM) images show that both the magnetic assisted impaction coater (MAIC) and the hybridizer (HB) produce particles that are significantly more uniformly coated than using either a V-shape blender or simple hand mixing. Image analysis confirms that MAIC and HB provide higher surface coverage for the amount of guest material (flow aid) used. The improvement in flowability of coated cornstarch was determined from angle of repose measurements using a Hosokawa powder tester. These measurements show that nanosized silica provides advantageous levels of flowability enhancement, whereas mono-dispersed 500 nm silica did not improve the flow properties of cornstarch. This experimental result is consistent with a theoretical derivation based on the original Rumpf model, which shows that flowability improvements are inversely proportional to the guest particle size for a given host particle size or size of surface asperities. Experimental results also indicate that surface treated hydrophobic silica is more effective in improving the flowability of cornstarch particles than untreated hydrophilic silica. An increase in processing time using MAIC and the V-blender also improves the flowability of the cornstarch since the guest particles are more deagglomerated and better dispersed, the longer the processing time.

Advantageous Downstream Processing of Dry Coated Cohesive Host Particles

The dry coating techniques described herein, wherein cohesive host particles/powders are dry coated with an effective level of nanosized guest particles, yield particle/powder systems that demonstrate enhanced and/or improved downstream processing properties. For example, the dry coated host particles/powders of the present disclosure demonstrate enhanced fluidization and film coating/granulation properties. Additional advantageous downstream processing benefits associated with the disclosed dry coated cohesive particles/powders will be readily apparent to persons skilled in the art from the description provided herein of exemplary advantages/benefits.

A. Downstream Fluidization of Dry Coated Cohesive Powders/Particles

Fluidization is widely used in powder processing and offers several powder processing advantages, e.g., continuous powder handling capabilities and desirable levels of gas-solid contact. Generally, effective fluidization translates to good mixing, high heat and mass transfer coefficients and high rates of reaction. However, particles with different physical properties have very distinct fluidization behaviors. As is well known to persons skilled in the art, based on empirical observations, Geldart classified powders into four groups depending on their size and the density difference between the solid particles and the fluidizing gas, i.e., Groups A, B, C and D. The distinction between these powder groupings relates to different fluidization behaviors. Thus, for example, in a conventional gravity-driven fluidized bed, particles having an average diameter smaller than 20 microns behave as and are characterized as Geldart Group C powders. Similarly, larger particles, e.g., particles having a diameter of 30-40 microns, also behave as Geldart Group C powders when they have densities of less than 1000 kg/m³. These powders are extremely difficult to fluidize and generally will form cracks, channels or “rat holes”. In some cases, the entire bed lifts as a solid plug when exposed to fluidizing gas.

Generally, the behavior of Group C powders is due to the very large interparticle forces that exist as the particle size drops below 20 microns. These interparticle forces increase as the surface-to-volume ratio of the particles becomes larger, and the distance between the particles becomes smaller. Thus, Geldart Group C powders are difficult to fluidize because of the strong cohesive forces between the particles, which may significantly exceed the external mechanical forces to which they are subjected in connection with the fluidization process.

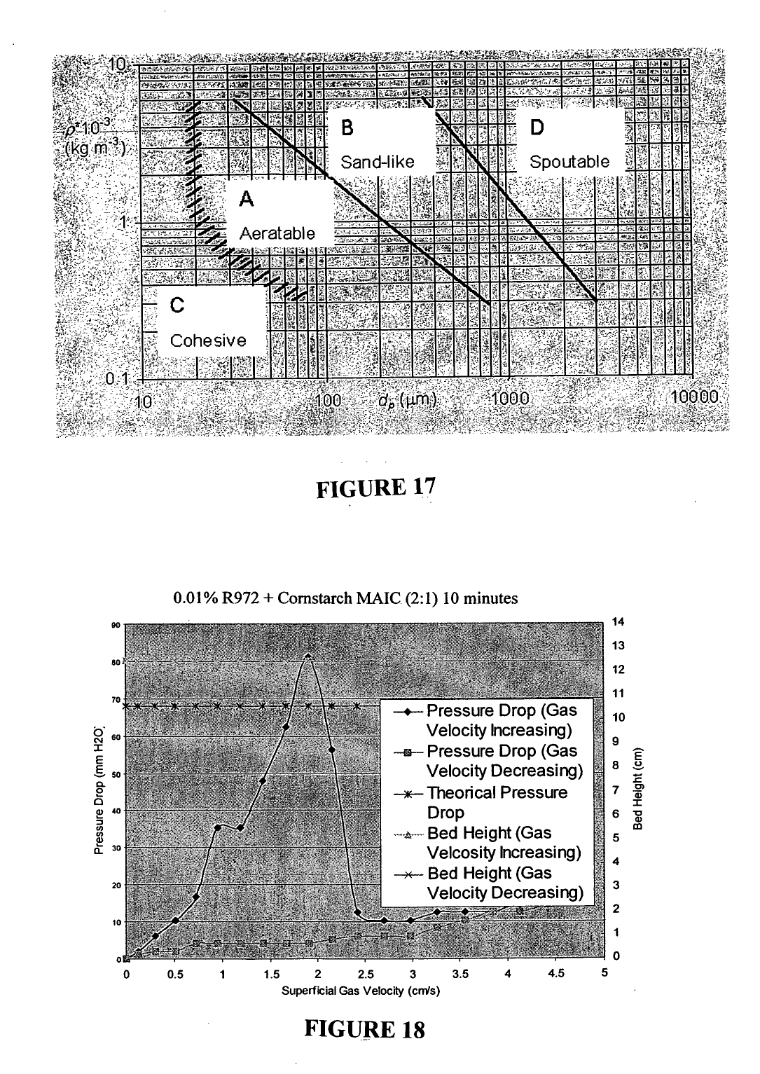

According to the present disclosure, dry coating of the cohesive particles/powders, e.g., Group C powders, with nanosized guest particles (e.g., fumed silica), the interparticle forces are significantly reduced and fluidization of the dry coated powder/particles can be effectively and reliably undertaken. With reference to FIG. 17, a graphical representation of the Geldart classifications is provided. As shown in FIG. 17, a boundary exists between Geldart Class C and Class A powders. This boundary is generally associated with relative cohesive forces, body weights and other forces (such as drag forces on the particles). As the cohesive forces are reduced (without reducing the particle weight), the boundary between the Class C and Class A particles moves towards the left of FIG. 17, indicating smaller and smaller particles can now be fluidized as Class A particles.

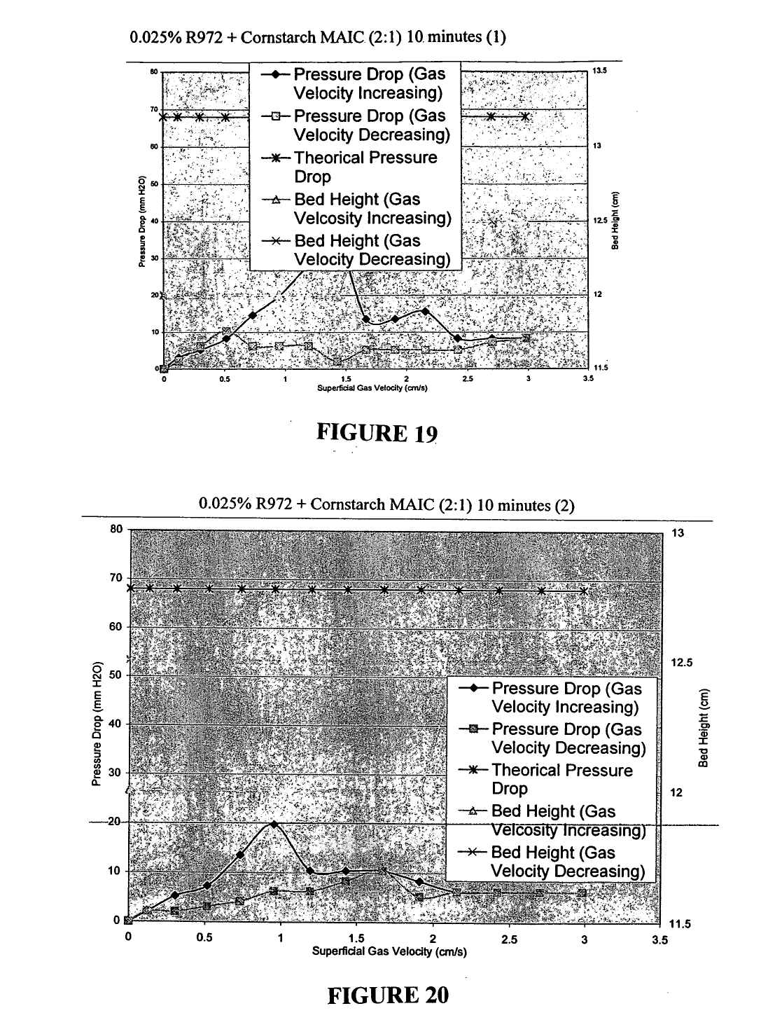

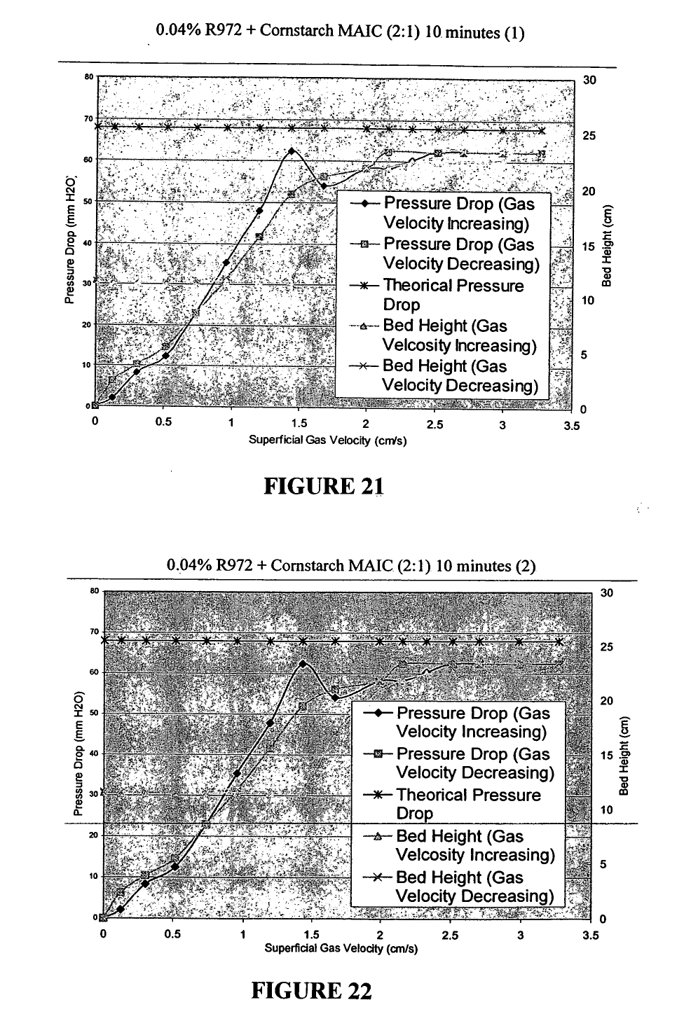

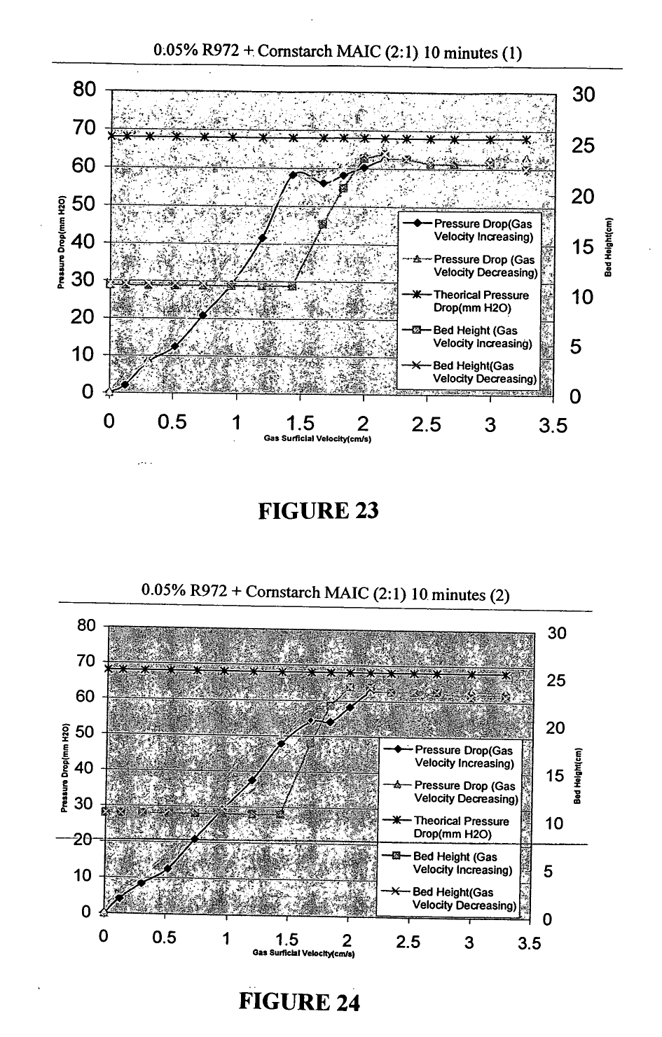

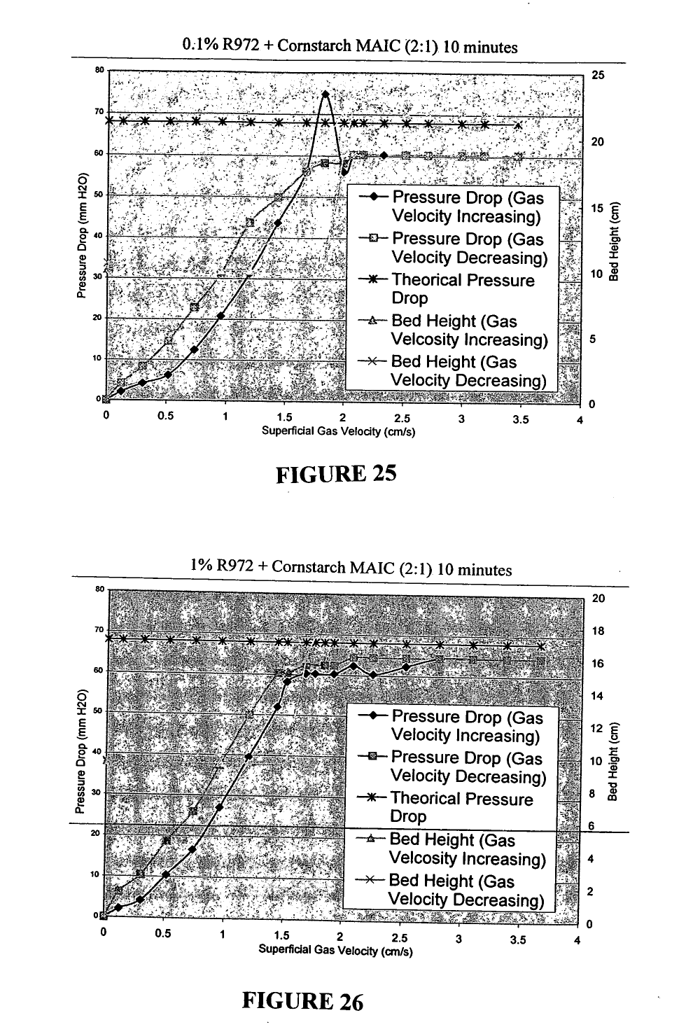

A series of experimental tests were conducted with dry coated particles according to the present disclosure. The results of these tests are set forth in the plots of FIGS. 18-26. As shown by the test results set forth in such figures, dry coating of nanosized guest particles at levels of less than 0.04 wt % are not effective in achieving fluidization of Class C cohesive powders. The ineffectiveness at such reduced coating levels are reflected in the curves of FIGS. 18-20, where the pressure drop does not equal the theoretical constant value, which is equivalent to the weight of the powder bed. However, as the dry coating level of nanosized particles is increased above 0.04 wt %, excellent fluidization occurs, as determined both visually and as indicated by the pressure drop profiles of FIGS. 21-26.

Based on the results reported herein, the present disclosure advantageously permits effective fluidization of Class C powders with limited pre-processing, using conventional fluidization equipment and techniques. Indeed, the test results reported herein demonstrate that a cohesive host powder, i.e., cohesive cornstarch particles, can be stably fluidized in a conventional vertical fluidized bed without any additional external forces such as vibration or sound waves. The pre-treatment step involves a dry coating of the cohesive cornstarch particles with nanosized guest particles, e.g., R972 silica. According to the disclosed cornstarch/R972 silica system, a threshold level of 0.04 wt % silica is required for effective fluidization to be achieved. Different threshold levels may be encountered for different host/guest particle systems, as will be readily apparent to persons skilled in the art.

Bed expansion levels of approximately two (2) times the initial bed height were advantageously achieved with the dry coated cornstarch powders disclosed herein. Additional levels of R972 silica had no significant effect on minimum fluidization velocity required to achieve desirable fluidization levels, or on overall bed expansion. Although the experimental results reported herein involved a vertical fluidized bed system, advantageous results can also be achieved with alternative fluidization systems, e.g., a Wuster rotating fluidized bed system (Yenchen Machinery Co., Ltd.; Taipei, Taiwan).

The fluidization results described herein are highly advantageous and permit a host of fluidization processes to be undertaken with dry coated host particles that would otherwise exhibit cohesive forces that would prevent effective fluidization, e.g., fluidized-bed based coating processes, reactions, synthesis and the like. Because the dry coated cohesive powders generated according to the present disclosure are stable (robust coating), i.e., the guest particles are securely adhered to the host particles, the present disclosure is particularly effective for subsequent unit operations, e.g., fluidization, because the advantageous properties associated with the dry coated powders/particles are not lost in transit to or processing in the subsequent unit operation.

B. Downstream Coating of Dry Coated Cohesive Powders/Particles

As described herein, dry coating of cohesive host powders with appropriate levels of nanosized guest particles is effective to facilitate reliable and advantageous fluidization of the cohesive powders. Through stable fluidization of the cohesive powders, it is possible to coat or granulate such cohesive powders, e.g., by spraying the fluidized particles with a desired binder solution to coat or granulate the cohesive particles. Thus, the advantageous dry coating process described herein greatly facilitates downstream processing of the underlying cohesive powders.

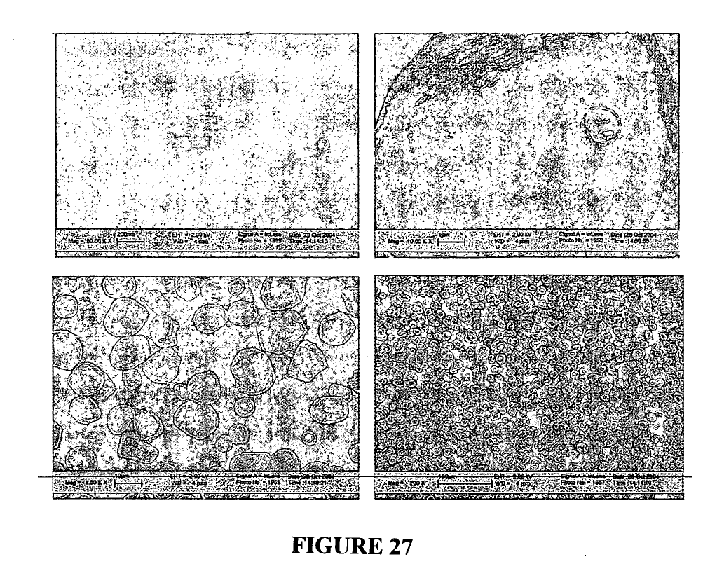

In an exemplary implementation of the present disclosure, HPC coatings (hydroxypropyl cellulose, a water soluble polymer; average MW 100,000) were coated on fluidized cornstarch (host particle) that had been dry coated with nanosized silica (guest particle) as described herein. With reference to FIG. 27, SEM images (at different magnifications) show a 1.0 wt % coating of HPC onto a dry coated cornstarch host powder system according to the present disclosure. The HPC coating was filmily applied to the fluidized cornstarch without agglomerate formation. The precoated fumed nanosized silica particles, i.e., the guest particles that were dry coated onto the cornstarch host particles, were completely covered by the HPC film. Moreover, the HPC coated particles advantageously remained distinct, non-agglomerated, individual particles.

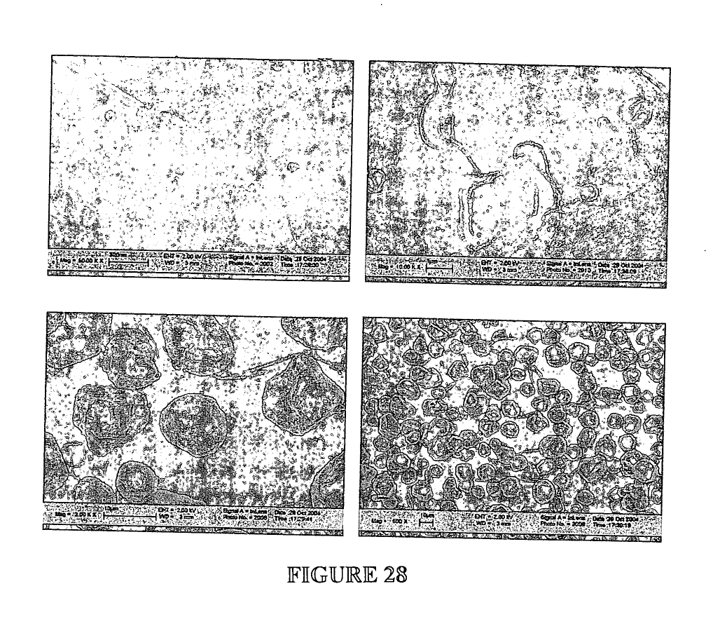

Turning to FIG. 28, coated particles formed through a further exemplary implementation of the present disclosure are shown in SEM images of different magnifications. The particles depicted in FIG. 28 were formed through a 2.0 wt % application of HPC coating to fluidized cornstarch particles that had been dry coated with nanosized silica according to the present disclosure. According to this exemplary implementation, 15 micron cornstarch particles were effectively coated with 2.0 wt % HPC coating in a commercially available fluidized bed, namely a Mini-Glatt fluidizer (Glatt Maschinen & Apparatebau AG; Pratteln, Switzerland). The coated particles maintained their individual/distinct character without agglomerate formation.

In a further exemplary implementation of the disclosed coating technique, 5-10 micron aluminum particles (host) that had been dry coated with nanosized guest particles were coated with a film of polymeric material. As with the cornstarch particles described above, the polymer-coated aluminum particles remained as individual/distinct particles without agglomerate formation.

C. Downstream Granulation of Dry Coated Cohesive Powders/Particles

According to further exemplary implementations of the present disclosure, cohesive host particles that had been dry coated with nanosized guest particles were advantageously granulated in a fluidized bed system. Thus, in a first “bottom spray” granulation system, dry coated cornstarch particles were advantageously granulated according to the following granulation conditions:

| | |
|---|---|
| Dry air temperature: | 65° C. |
| Fluidize air pressure | 0.07 Bar |
| Atomization air pressure | 0.07 Bar |
| Spray rate | ˜0.7 g/min |
| Spray solution | 2.0 wt % HPC water solution |
| Raw material | 1.0 wt % A200 + cornstarch; 95.25 g |
| Processing time | ˜150 mins |
| Binder ratio | 2.10 wt % |

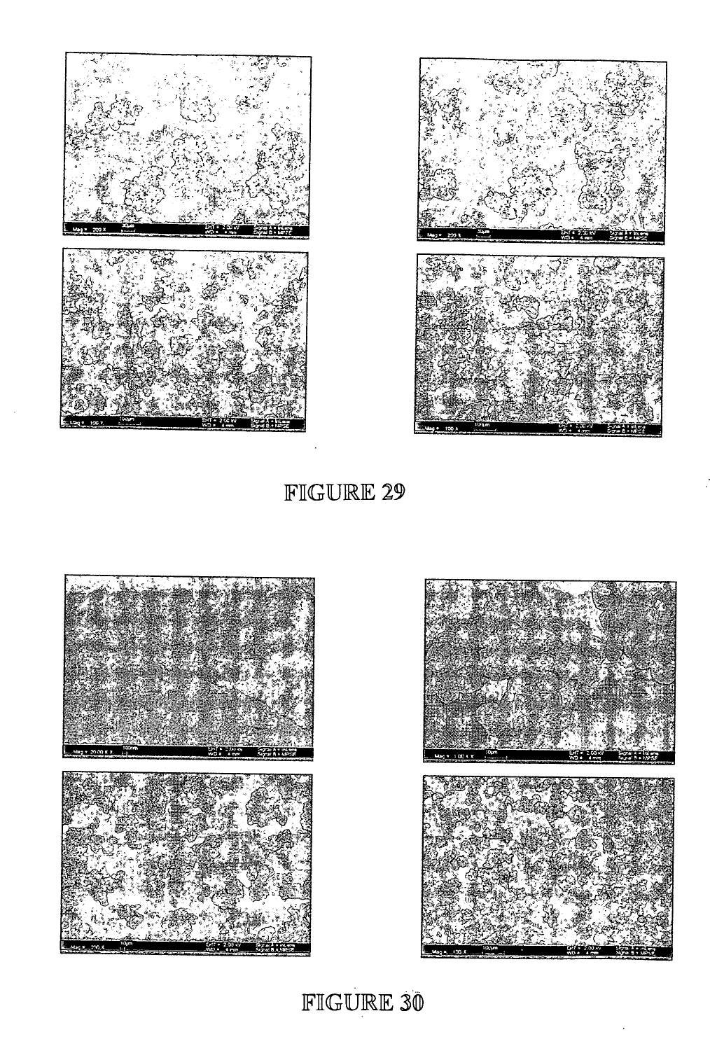

As shown in the SEM images of FIG. 29 (200× and 100×), the dry coated cohesive cornstarch particles were successfully granulated into granules having an average particle size of about 100 microns.

In a further exemplary implementation of the present disclosure employing a “top spray” granulation system, dry coated cohesive cornstarch particles (approximately 15 microns) were advantageously granulated according to the following granulation conditions:

| | |
|---|---|
| Dry air temperature: | 65° C. |
| Fluidize air pressure | 0.08 Bar |
| Atomization air pressure | 1.0 Bar |
| Spray rate | ˜1.1 g/min |
| Spray solution | 4.0 wt % HPC water solution |
| Raw material | 0.5 wt % A200 + cornstarch; 80.0 g |
| Processing time | ˜60 mins |
| Binder ratio | ˜3.40 wt % |

As shown in the SEM images of FIG. 30 (various magnifications), the dry coated cornstarch particles produced according to the present disclosure were effectively granulated by the exemplary “top spray” granulation technique described herein, yielding granulated cornstarch particles having an average particle size of about 100 microns.

Density is Not an Indicia of Fluidization Potential

As demonstrated below, apparent density (or packing density or what may be called tapped density) is not a controlling factor in determining or predicting the fluidization behavior of powder systems. These results are inconsistent with prior art teachings and further demonstrate that the disclosed robust coating techniques achieve desirable results in an altogether different and unexpected way.

For purposes of the experimental results reported herein, cornstarch was employed as the host powder. Cornstarch is lighter than two different guest materials that were employed in this experimental work in terms of packing density. As shown herein, the same percentage of coating in MAIC and V-Blender systems does not provide similar fluidization results. In fact, the V-blender mixed product (which correlates with prior art mixing techniques) did not fluidize.

PROPERTIES OF HOST AND GUEST PARTICLE

| | Material | | |
|---|---|---|---|
| | Density | Packing Density | |
| | (g/cm³) | (g/cm³) | Size |
| Cornstarch | 1.550 | 0.806 | 15 μm |
| TiO₂ (R104) | 4.69 | 1.375 | ˜220 nm |
| Nickel | 8.9 | 1.7 | 80˜150 nm |

Three powder systems were tested: (1) Cornstarch coated with 5% Nickel by MAIC, according to the present disclosure, (2) Cornstarch coated with 1.5% TiO₂ (R104) by MAIC according to the present disclosure, and (3) Cornstarch blended with 1.5% TiO₂ (R104) by V-blender. The noted powder systems were tested in a conventional fluidized bed.

1) Cornstarch Coated with 5% Nickel by MAIC

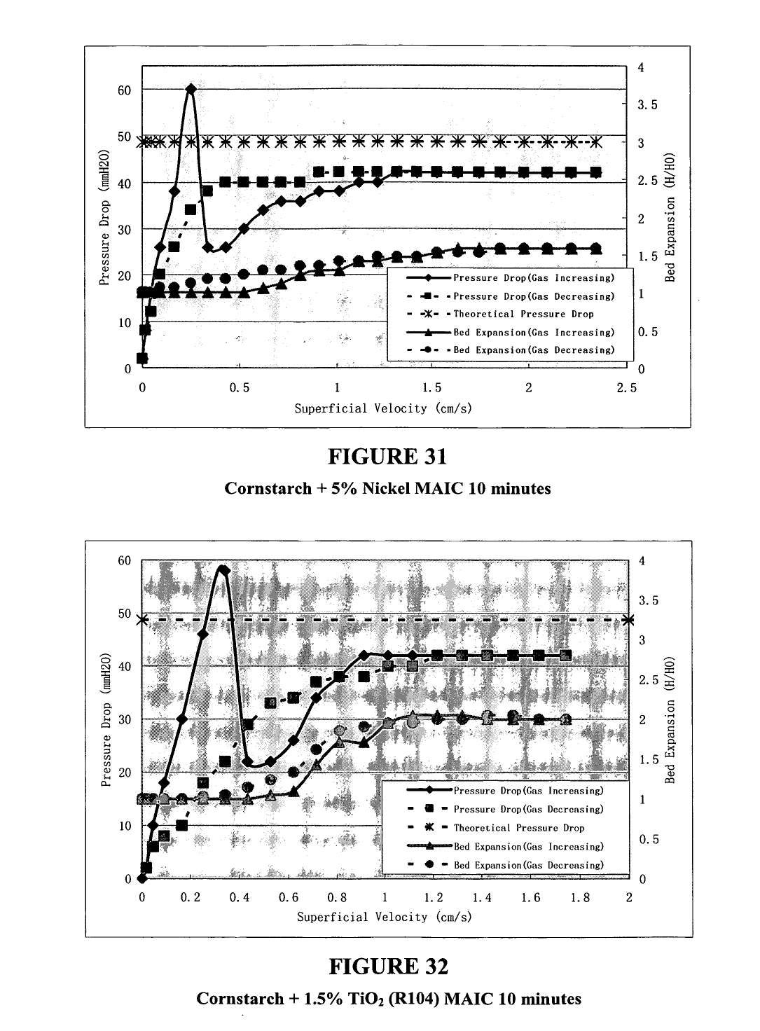

The measured pressure drop and bed expansion are plotted in FIGS. 31-32. These figures demonstrate that pressure drop rose with the superficial velocity until it reached a maximum value. Upon breaking up the interaction of the particles, the pressure drop decreased because of channeling. Finally, the plots of pressure drop reach a plateau when the particles are stably, i.e., effectively, fluidized.

2) Cornstarch Coated with 1.5% TiO₂ R104 in MAIC

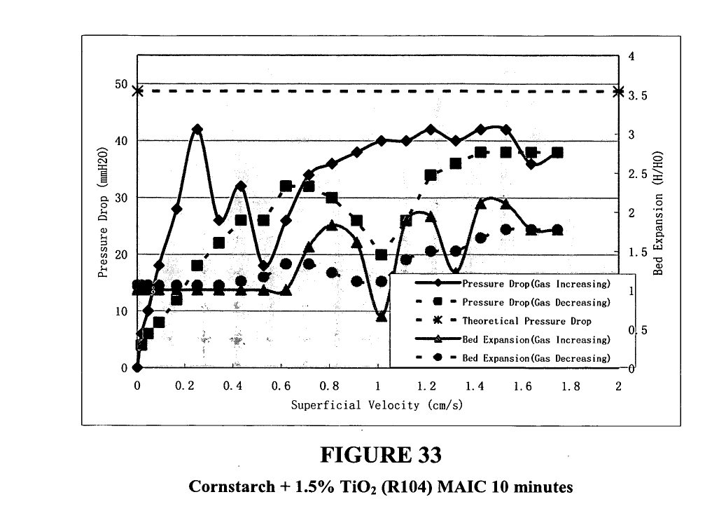

As shown in FIG. 33, the fluidization behavior of cornstarch coated with 1.5% TiO₂ (R104) according to the present disclosure was similar to that of cornstarch coated with 5% nickel, i.e., the powder was stably, i.e., effectively, fluidized.

3) Cornstarch Blended with 1.5% TiO₂ R104 using a V-blender.

Unlike the 1.5% TiO₂ coated cornstarch systems discussed above, cornstarch blended with 1.5% TiO₂ (R104) exhibited constant channeling. Fluidization air passed through the channels and, as a result, the pressure and bed expansion could not obtain a stable status. Thus, effective fluidization could not be achieved.

As demonstrated in the foregoing experimental results, robust coating pursuant to the present disclosure is effective in producing powder systems that may be reliably and efficiently fluidized, whereas prior art blending techniques are ineffective in achieved desired fluidization results.

Coating and Granulation of Fine Particles Using a Conventional Fluidized Bed

As noted previously, fluidized beds are widely used for particle coating and granulation in various industries, e.g., the pharmaceutical and chemical industries, at least in part based on the high heat/mass transfer rates and ease of scale-up associated with fluidized bed systems. However, conventional fluidized beds cannot be used for handling fine particles (less than 40 microns) due to their poor fluidization behavior. As further demonstrated below, nanosized particles may be used to create nano-scale roughness on the surface of particles that are less than 40 microns in diameter, thereby advantageously improving the fluidization behavior of cohesive powders. Experimental runs utilizing the disclosed technique were performed on fine particles (15 micron cornstarch and 5-15 micron aluminum) using a conventional gravity-driven fluidized bed unit. The results demonstrate that fine cohesive particles can be homogeneously fluidized after they are pre-treated with nanosized particles. Subsequently, the pre-treated particles can be further processed, e.g., uniformly film-coated in commercially available Wurster-type fluidized bed coaters. By changing the operating conditions, the same process can be used for granulation of fine particles.

The properties of the experimental materials utilized herein are set forth in the following table.

Properties of Particles

| Material | Particle Size | Density (kg/m³) | Surface Properties |
|---|---|---|---|
| Cornstarch | 15 μm | 1550 | Hydrophilic |
| Aluminum | 15 μm | 2700 | Hydrophilic |
| Fumed Silica | ˜20 nm | 2650 | Hydrophilic |
| (EH-5) | | | |

The fine powders, cornstarch (from Argo Company) and aluminum (from Alfa Aesar Company), were first dry coated with 0.5 wt % of nanosized fumed silica EH-5 according to the dry coating procedures described above. An aqueous polyvinylpyrrolidone (PVP) solution (with average molecular weight of 40,000) was used as a binder for the granulation of the cornstarch particles. Eudragit E100 (Degussa) dissolved in acetone or acetone-water was used as a coating polymer for the aluminum particles. Both the granulation and coating processes were conducted in a standard fluidized bed with a biaxial nozzle top spraying system and without a Wurster tube.

A LS 230 Coulter particle size analyzer was used to measure the particle size distribution of the granulated cornstarch and the coated aluminum powders. The Hausner index (ratio of packing density to bulk density) and angle of repose of the granulated particles were characterized by a Hosokawa Powder Tester (PT-N). A LEO 1530VP field emission scanning electron microscope was used to characterize the particles at microscale.

1) Granulation of Cornstarch Particles Using a Fluidized Bed

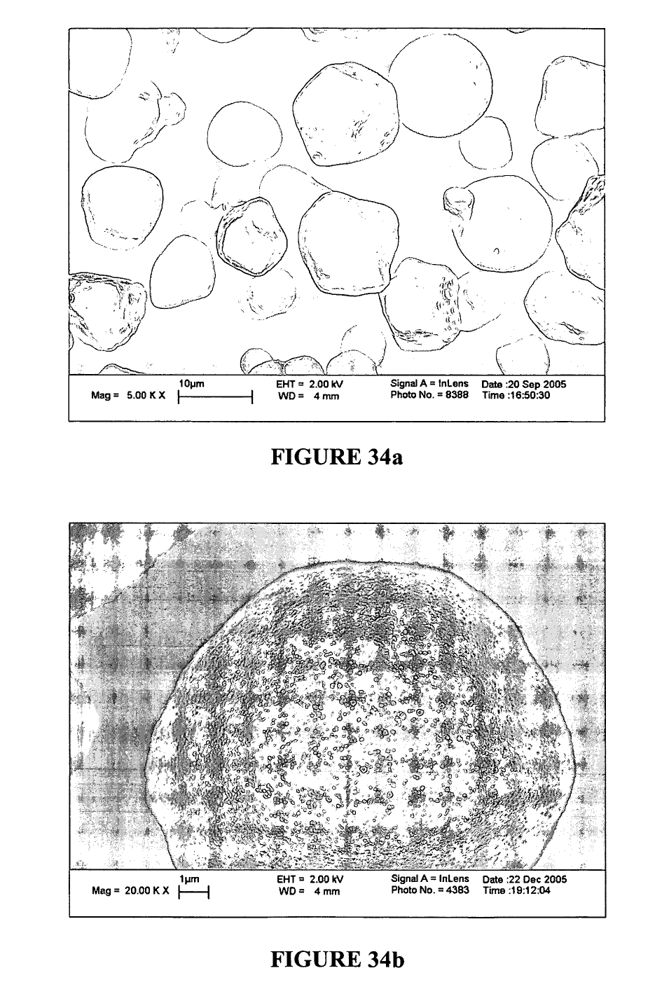

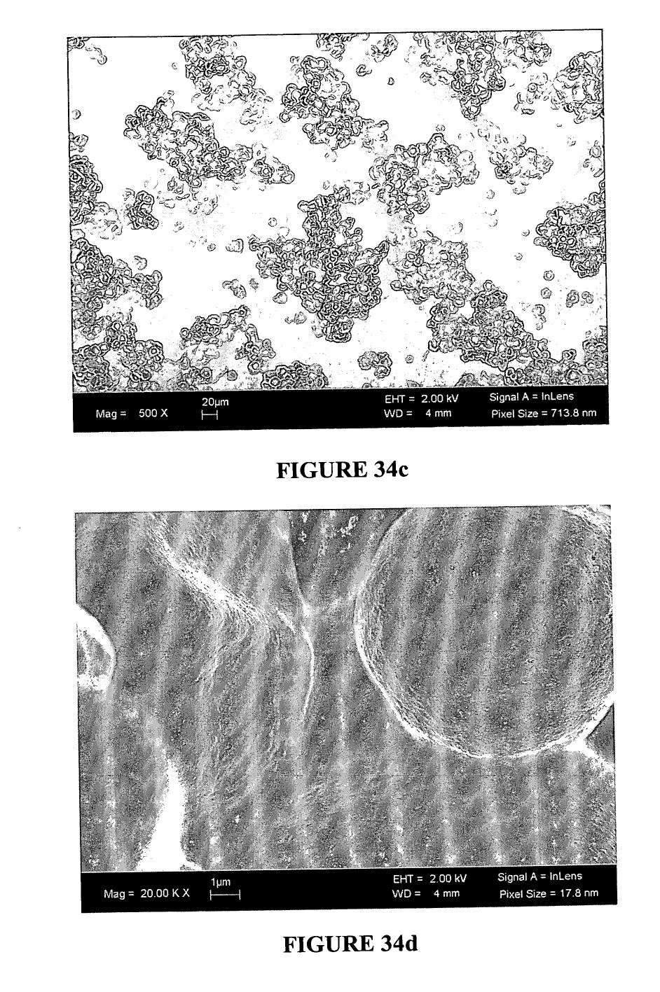

The experimental results for granulation of the cornstarch particles in the fluidized bed are set forth in Table 4 (appended hereto) for a number of different experimental runs. Typical SEM pictures of pure cornstarch and the granules are shown in FIG. 34. Through the dry coating process, the cornstarch particles (FIG. 34a) were evenly coated with nanosized silica EH-5 particles (FIG. 34b), which significantly improved the flowability of the original cornstarch particles as indicated by the much lower Hausner index and angle of repose of the nanocoated particles as listed in Table 4. It is noted that the Hausner index for pure cornstarch is 1.57 and its angle of repose is 47.2 degrees. FIGS. 34c and 34d are typical SEM images of the granules generated according to the present disclosure, and such images clearly show the loose structure as well as the polymer binding between the granulated particles.

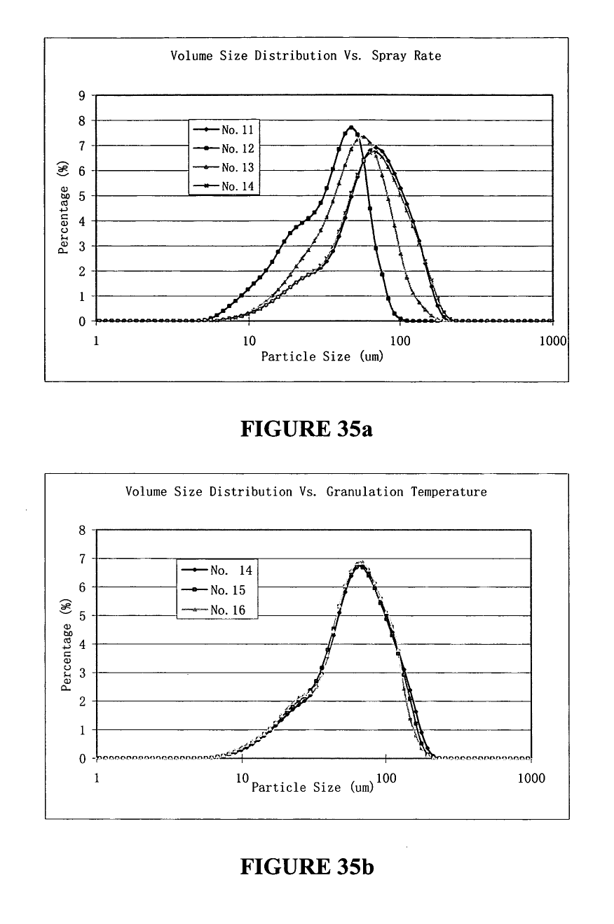

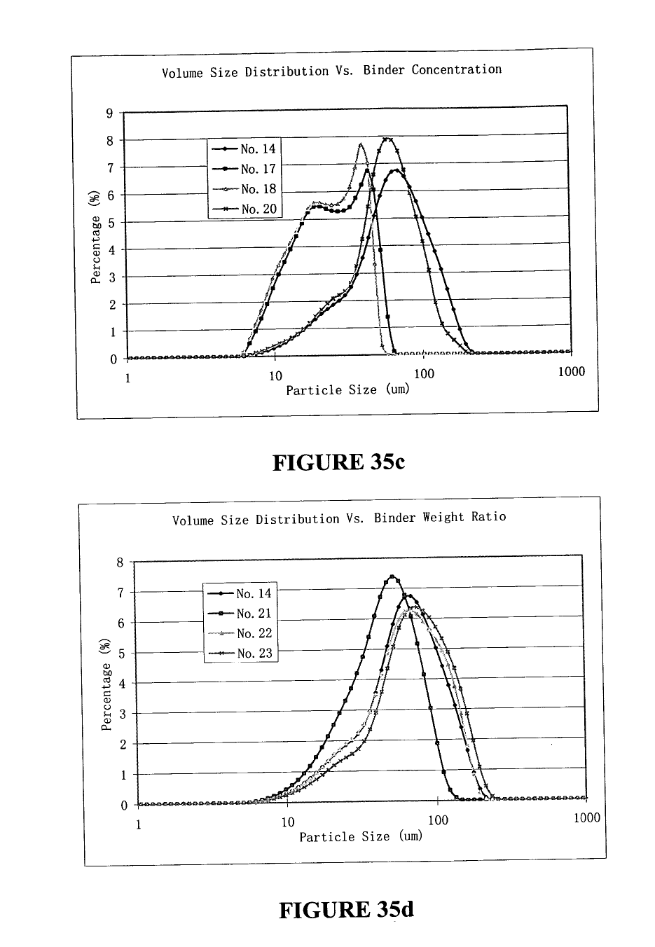

The effect of varying the operating parameters on granulation results are illustrated in the plots of FIG. 35. As set forth in Table 4 and shown in FIG. 35a, the mean size of the cornstarch granules increased from 38.6 μm to 72.7 μm when the spray rate increased from 0.91 ml/min to 2.11 ml/min. Of note, further increases in the spray rate from 2.11 ml/min to 2.57 ml/min did not show a further increase in granule size. The inlet air temperature (ranging from 40 to 80° C.) did not effect granule size (FIG. 35b), which may be attributed to the small amount of inlet air flux. These results are in agreement with bed temperature variations detected during the experimental runs, which only showed a few degrees difference as the inlet air temperature increased from 40 to 80° C.

The effect of binder concentration on the granulation is shown in FIG. 35c. The average granule size increased from 63.4 μm to 72.7 μm when the binder (PVP) concentration increased from 4.0 wt % to 8.0 wt %. However, further increases in the binder concentration to 12.0 wt % or 16.0 wt % decreased the granule size down to 26.2 or 28.3 μm. This behavior may be attributed to faster drying of the polymer binder at these elevated levels. FIG. 35d shows that the granule size is increased by increasing the binder amount (binder to powder weight ratio) due to the stronger adhesion force between particles. The largest granule size is 82.9 μm when the binder concentration is 8.0 wt %.

2) Coating of Aluminum Particles Using the Fluidized Bed

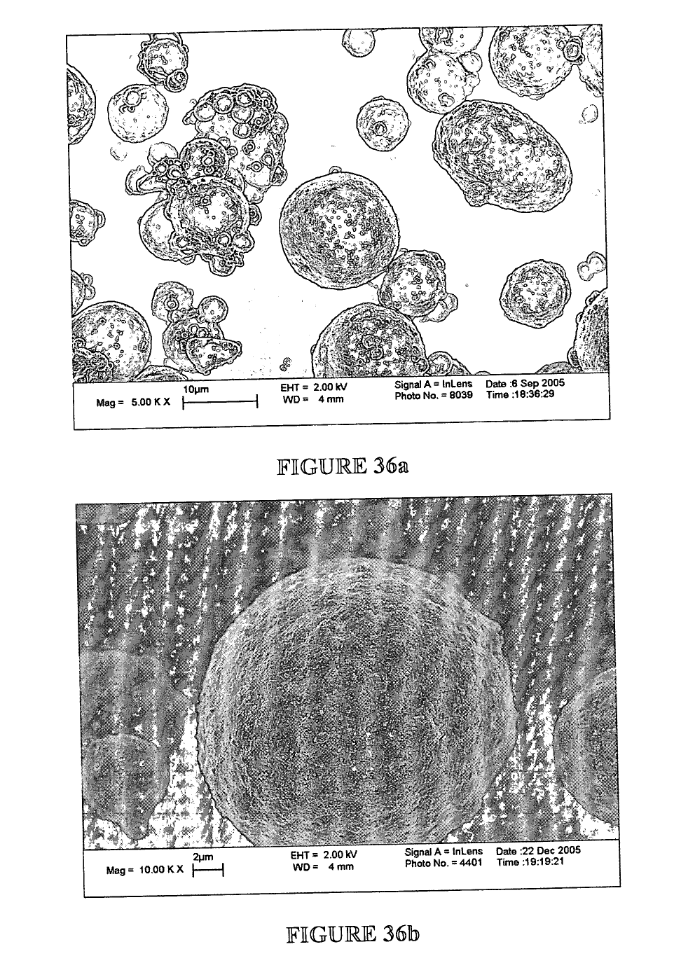

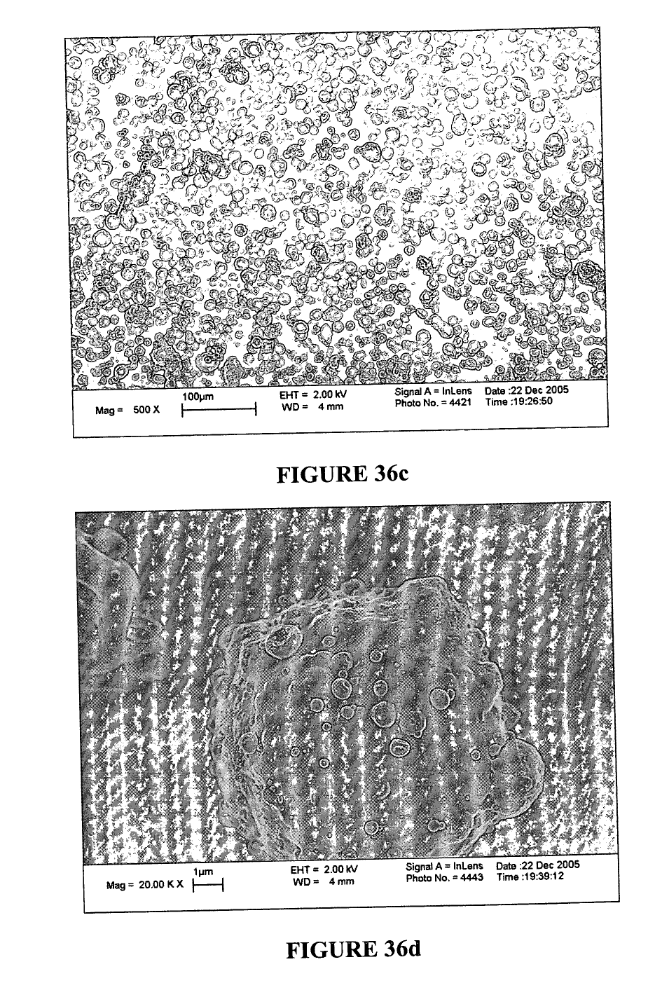

In further experimental runs according to the systems and methods of the present disclosure, 15 micron (mean size) aluminum particles were dry coated and granulated as described herein. The operating conditions and results for the coating experiments of the aluminum particles in a fluidized bed are set forth in Table 5 appended hereto. In addition, typical SEM micrographs of raw aluminum and the coated particles are set forth in FIGS. 36a 36d. The SEM images show that the fine aluminum particles are evenly dry coated with nanosized particles first (see FIG. 36b), and then by a Eudragit E100 polymer film in the fluidized bed (FIG. 36d). In addition, it can be seen in FIG. 36d that the coated aluminum particles are separate/individual, rather than being agglomerated.

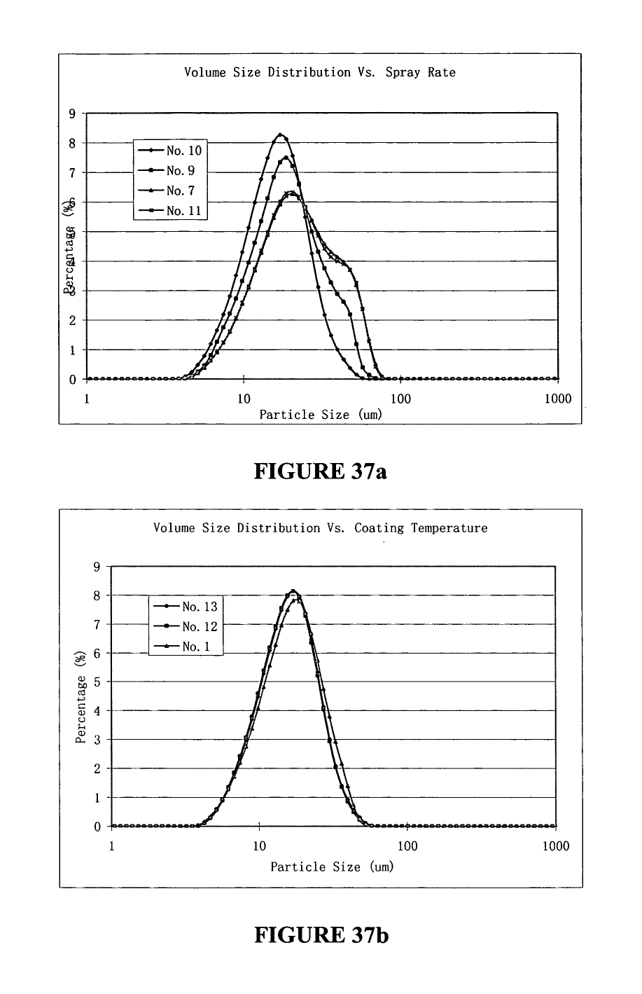

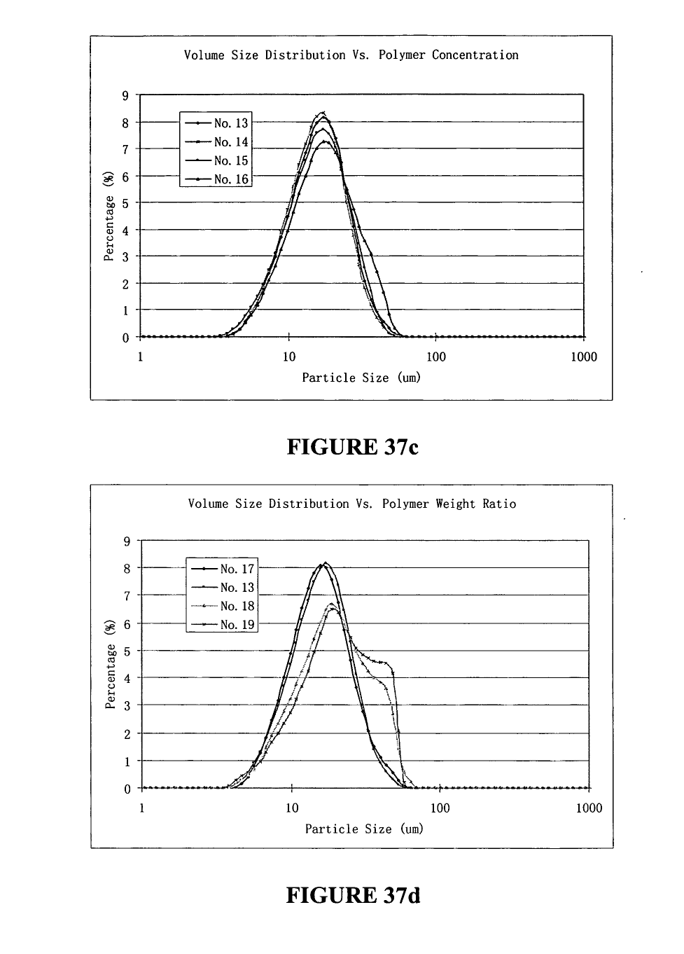

As shown in FIG. 37a, by increasing the spray rate from 0.91 ml/min to 3.01 ml/min, the coated particle size slightly increased from 18.4 μm to 26.6 μm due to some agglomeration of the coated particles. As was found in the granulation experiments, the inlet air temperature is relatively insensitive to the coated particle size as shown in FIG. 4b. The same insensitivity is also found for the polymer concentration since the coated mean particle size only varies from 17.6 to 20.2 μm as shown in FIG. 4c. However, changing the polymer wt % ratio from 2 to 8 did increase the coated particle size from 18.1 μm to 24.4 as shown in FIG. 4d.

Thus, as shown herein, granulation and coating of cohesive particles (e.g., cornstarch and aluminum) may be successfully performed in a conventional fluidized bed after dry coating the cohesive particles with nanosized particles. The experimental results set forth herein indicate that the granule size may be significantly increased by increasing the spray rate and the binder ratio, while the inlet air temperature has a lesser effect on the mean size of the granules (perhaps due to insufficient amounts of inlet air flux in the experimental design). The amount of binder (ratio of polymer to particle weight) is also an important factor in determining and/or controlling granule size. The coating results advantageously demonstrate that the pre-coated aluminum particles (dry coated with nanosilica) are individually film coated with polymer in the fluidized bed with little agglomeration. The coating performance can be fine tuned by varying operating parameters, e.g., the spray rate and the amount of the coating polymer, to achieve desired end-products.

Effect of Smaller Host Particle Size

In addition to the experiments herein where 15 micron cornstarch and 15 micron aluminum host particles were dry coated with silica nanoparticles and then fluidized, three smaller sizes of aluminum powders (3-4.5 μm, 4.5-7 μm and 10-14 μm) were used as host particles to demonstrate the effect of host particle size on cohesion force reduction and fluidization behavior according to the present disclosure. All of the aluminum powders were dry coated using MAIC with different weight % of R972 silica nanoparticles corresponding to a theoretical surface area coverage of 100%, as shown in the table below.

Different Size Aluminum Powders with R972 Silica Nanoparticles

| | Weight Ratio | Theoretical | |
|---|---|---|---|
| | of Guest | Surface Area | Host Particle |
| Particle System | Particles | Coverage (%) | Size (μm) |
| Aluminum + R972 | 0.52 | 100 | 3˜4.5 |
| silica | 1.1 | 100 | 4.5˜7 |
| nanoparticles | 1.53 | 100 | 10˜44 |

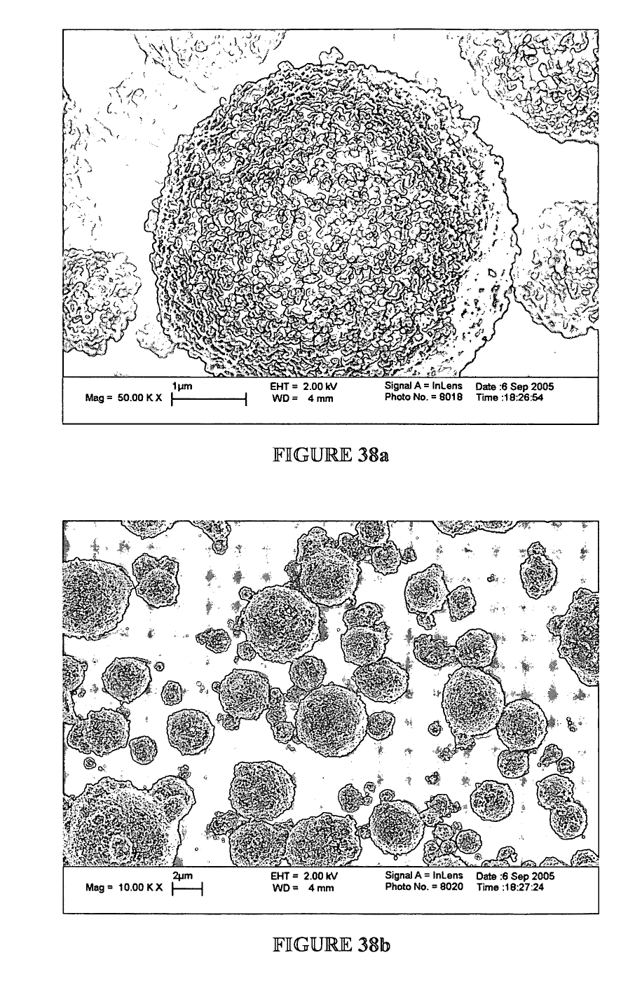

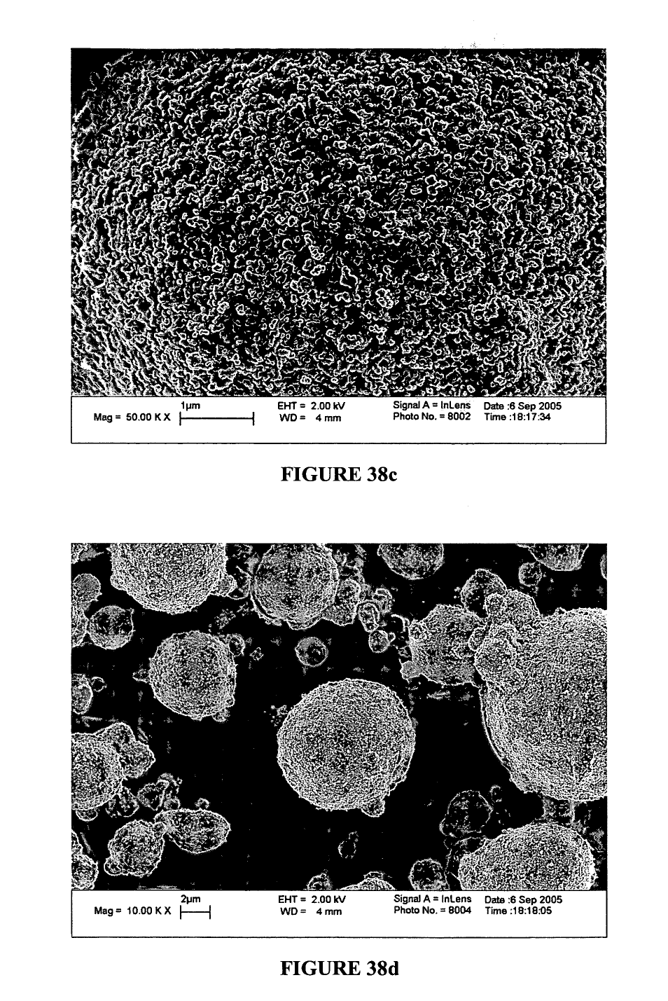

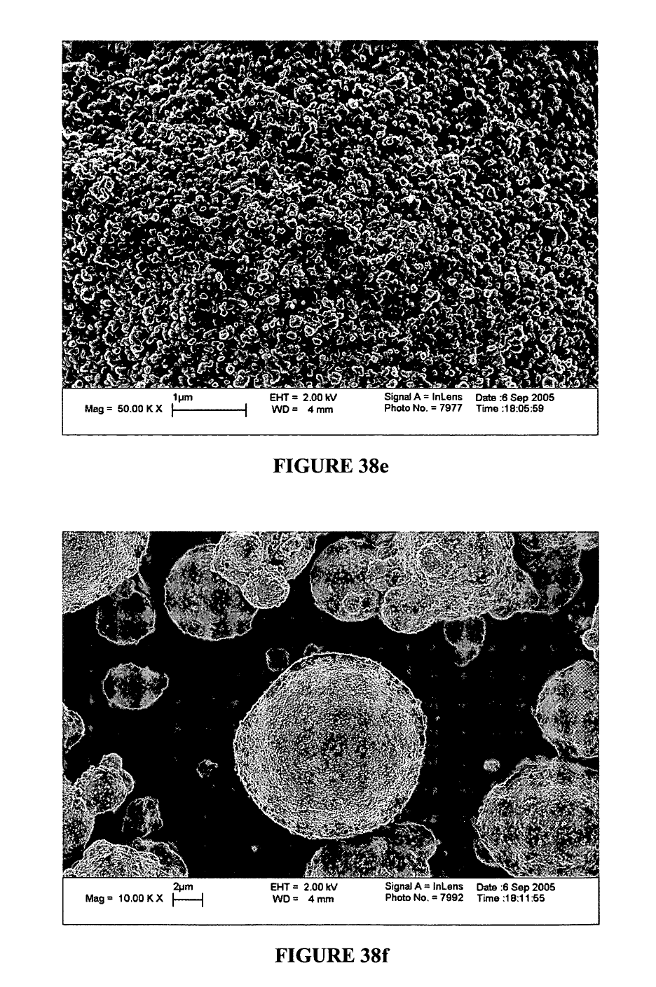

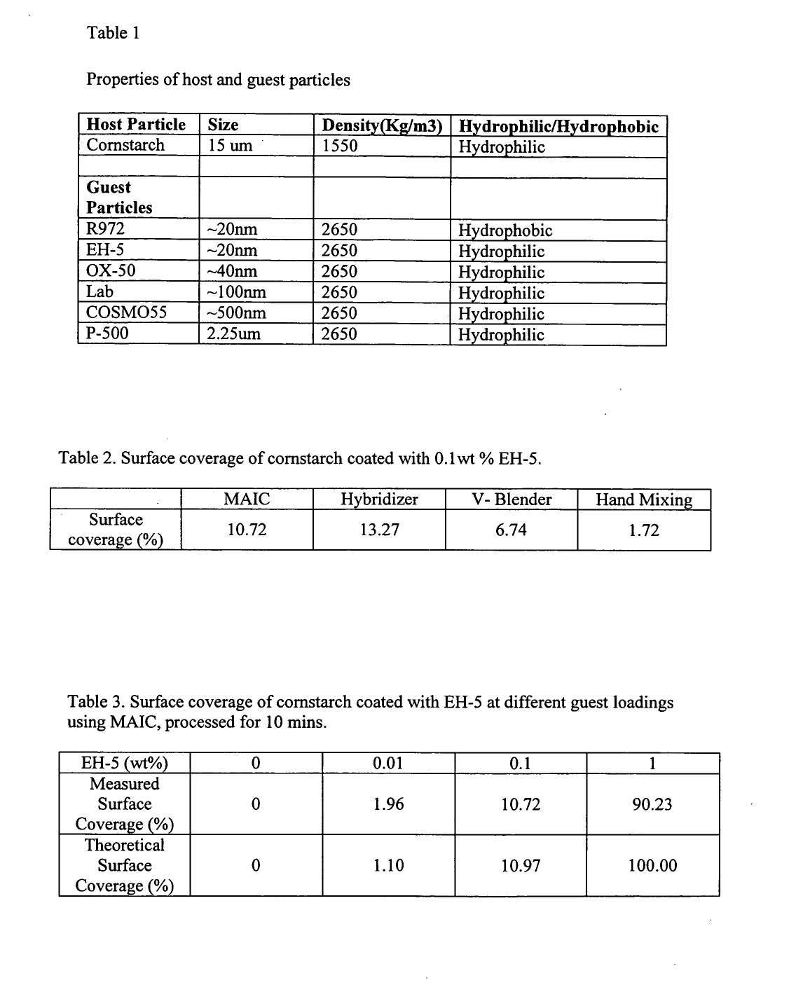

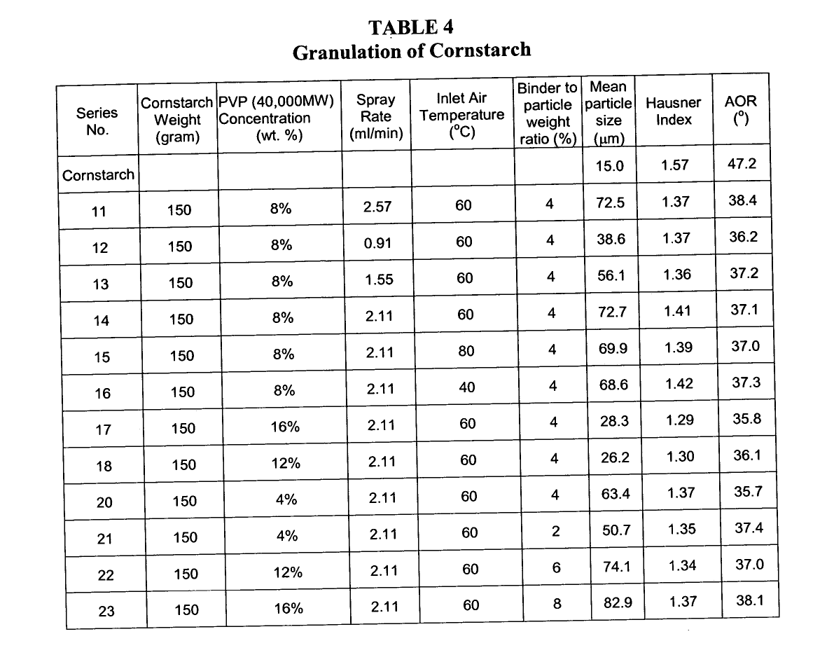

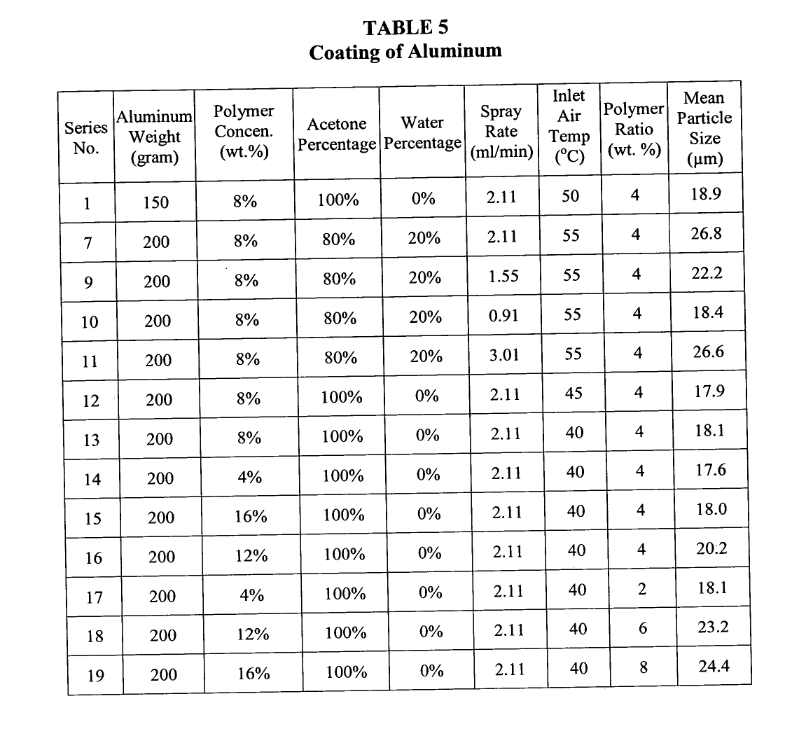

SEM images of the coated aluminum particles are shown in FIGS. 38a-38f and, as seen in such electron micrograph images, the surfaces of all the aluminum particles were coated with silica. After coating, the aluminum particles were fluidized in a conventional fluidized bed and it was observed that the coated 4.5-7 micron and the coated 10-14 micron aluminum particles fluidized well. However, in this preliminary testing, the smaller 3-4.5 micron coated aluminum particle systems showed channeling and fluidized poorly, demonstrating typical Geldart Group C fluidization behavior. Refinements to the processing parameters may overcome this initial ineffectiveness for aluminum host particle sizes in the 3-4.5 micron range.

Thus, dry coating aluminum host particles with an average size of about 4.5-7 microns and 10-14 microns with R972 silica nanoparticles at a theoretical surface area coverage of 100% advantageously results in particles that can be well fluidized according to the present disclosure. These results demonstrate, inter alia, that the disclosed techniques are effective for fluidization of smaller particles, including particles having a mean particle size of as small as 5 microns.

Thus, the present disclosure provides an advantageous and reliable method for processing of cohesive host particles/powders. The disclosed method/technique involves a dry coating of the host particles with an appropriate level of nanosized guest particles, such that the guest particles are firmly adhered to the host particles, thereby permitting effective downstream processing. Exemplary downstream processing to which the dry coated host particles may be subjected include fluidization, coating and/or granulation. The present disclosure advantageously permits fluidization of particles below 30 microns in size in conventional fluidized bed equipment, and coating/granulation of particles below 50 microns in conventional fluidized processes. Moreover, the disclosed dry coating methods/techniques permit such downstream processing without the need to modify and/or replace existing downstream equipment. Additional applications, benefits and advantages of the disclosed systems, methods and techniques will be readily apparent to persons skilled in the art, for example, the dry mixing of different species of fine particles that were initially dry coated in a fluidized bed. Thus, although the present disclosure makes reference to exemplary implementations and/or examples thereof (e.g., exemplary host/guest particle systems), the present disclosure is not limited to or by such exemplary implementation/examples. Rather, the disclosed systems, methods and techniques are susceptible to wide ranging applications, as will be readily apparent to persons skilled in the art, without departing from the spirit or scope of the present disclosure.

### REFERENCES

[1] O. Molerus, Effect of interparticle cohesive forces on the flow behavior of powders, Powder Technology, 20, 161-175 (1978).
[2] H. O. Kono, C. C. Huang and M. Xi, Function and mechanism of flow conditioners under various loading pressure conditions in bulk powders, Powder Technology, 63, 81-86 (1990).
[3] B. H. Kaya, J. E. Leblanc, D. Moxam, D. Zubac, The effect of vibration on the rheology of powders, International Powder and Bulk Solids Handling and Processing, 324-337, (1983).
[4] J. M. Elbicki, G. I. Tardos, The influence of fines on the flowability of alumina powders in test hoppers, Powder Handling & Processing, 10(2), 147-149, (1998).
[5] Jose Manuel Valverde, Antonio Casterllanos, Antonio Ramos and P. Keith Watson, Avalanches in fine, cohesive powders, Physical Review E, 62, 6851-6860 (2000).
[6] Michelle Ramlakhan, Chang-Yu Wu, Satoru Watano, Rajesh N. Dave and Robert Pfeffer, Dry particle coating using magnetically assisted impaction coatings: modification of surface properties and optimization of system operating parameters, Powder Technology, 112, 137-148 (2000).
[7] P. Singh, T. K. S. Solanky, R. Mudryy, R. Pfeffer and R. N. Dave, Estimation of coating time in the magnetically assisted impaction coating process, Powder Technology, Vol. 121, No. 2-3, 159-167 (2001).
[8] R. Pfeffer, R. N. Dave, D. Wei and M. Ramlakhan, Synthesis of engineered particulates with tailored properties using dry particle coating, Powder Technology, Vol. 117, pp. 40-67 (2001).
[9] Rajesh N. Dave, Chang-Yu Wu, Bodhisattwa Chaudhuri, Satoru Watano, Magnetically mediated flow enhancement for controlled powder discharge of cohesive powders, Powder Technology, Vol. 112, pp. 111-125 (2000).
[10] Renwei Mei, Hong Shang, James F. Klausner and Elizabeth Kallman, A contact mode for the effect of particle coating on improving the flowability of cohesive powders, Kona, 15, 132-141 (1997).
[11] Y. C. Zhou, B. H. Xu, A. B. Yu and P. Zulli, An experimental and numerical study of the angle of repose of coarse spheres, Powder technology, 125, 45-54 (2002).
[12] E. Teunou, J. J. Fitzpatrick, E. C. Synnott, Characterization of food powder flowability, Journal of Food Engineering, 39, 31-37 (1999).
[13] A. Santomaso, P. Lazzaro, P. Canu, Powder flowability and density ratios: the impact of granules packing, Chemical Engineering Science, 58(13), 2857-2874 (2003).
[14] Rumpf, Chemie-Ingenieur-Technik 46, 1-11 (1974).
[15] Huber, Wirth, Electrostatically supported surface coating of solid particles in liquid nitrogen for use in Dry-Powder-Inhalers, Powder Technology 134, 181-192 (2003)
[16] Eric W. van der Vegate and Georges Hadziioannou, Scanning force microscopy with chemical specifity: an extensive study of chemically specific tip-surface interactions and the chemical imaging of surface functional groups. Langmuir, 13, 4357-4368 (1997).

1. A method for altering the physical properties of a cohesive powder, comprising:
a. providing a cohesive powder characterized by cohesive forces that inhibit downstream processing thereof;
b. dry coating the cohesive powder with nanosized guest particles at a level effective to overcome said cohesive forces and enhance said downstream processing of the cohesive powder.

2. A method according to claim 1, wherein the dry coating of the cohesive powder is undertaken in equipment selected from the group consisting of a magnetic assisted impaction coating (MAIC) apparatus, a hybridizer (HB) and a V-blender (VB) apparatus.

3. A method according to claim 1, wherein the nanosized guest particles are dry coated with respect to the cohesive powder at levels between about 0.04-2.0 wt %.

4. A method according to claim 1, wherein the nanosized guest particles are hydrophilic.

5. A method according to claim 1, wherein the nanosized guest particles are hydrophobic.

6. A method according to claim 1, wherein the cohesive powder comprises individual cohesive particles, and wherein the dry coating of the cohesive particles is effective to coat a surface area of said cohesive particles.

7. A method according to claim 1, further comprising downstream processing of said dry coated cohesive powder.

8. A method according to claim 7, wherein said downstream processing includes fluidization of said dry coated cohesive powder in a fluidized bed.

9. A method according to claim 8, further comprising coating of said fluidized, dry coated cohesive powder.

10. A method according to claim 8, wherein the mean particle size of said dry coated cohesive powder is less than about 15 microns.

11. A method according to claim 8, wherein the mean particle size of said dry coated cohesive powder is about 5 microns.

12. A method according to claim 8, further comprising granulating of said fluidized, dry coated cohesive powder.

13. A method according to claim 12, wherein said granulation is effected by a top spray or a bottom spray of a binder material.

14. A dry coated cohesive powder produced by the method of claim 1.

15. A dry coated cohesive powder produced by the method of claim 1, wherein said dry coated cohesive powder is adapted for fluidization in a fluidized bed.

16. A method for fluidizing a cohesive powder, comprising:
a. dry coating the cohesive powder with nanosized guest particles to define dry coated particles;
b. fluidizing the dry coated particles in a fluidized bed.

17. A fluidization method according to claim 16, wherein the cohesive powder has a mean diameter of less than 50 microns.

18. A fluidization method according to claim 16, further comprising granulating or film coating the fluidized, dry coated particles.

19. A fluidization method according to claim 16, wherein the nanosized guest particles are dry coated onto the cohesive powder at a level of about 0.04 to about 2 wt %.

20. A fluidization method according to claim 16, wherein the cohesive powder behaves as a Geldart Group C powder prior to dry coating and behaves as a Geldart Group A powder after dry coating thereof.
